(19) United States
(12) Patent Application Publication
(10) Pub. No.: US 2007/0186301 A1
(43) Pub. Date: Aug. 9, 2007

(54) Mutant BDNF gene introducing knockin mouse

(75) Inventors: Masami Kojima, Ikeda-shi (JP); Tomoko Hara, Ikeda-shi (JP); Hisatsugu Koshimizu, Ikeda-shi (JP); Megumi Kashihara, Ikeda-shi (JP); Takahisa Taguchi, Ikeda-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NATIONAL INSTITUTE OF ADVANCED INDUSTRIAL SCIENCE AND TECHNOLOGY, Tokyo-to (JP)

(21) Appl. No.: 11/649,360

(22) Filed: Jan. 4, 2007

(30) Foreign Application Priority Data
Jan. 5, 2006 (JP) 2006-000397
Apr. 20, 2006 (JP) 2006-116863
Jun. 6, 2006 (JP) 2006-156879

(51) Int. Cl. A01K 67/027; C12N 15/09

(52) U.S. Cl. 800/18; 435/320.1

(57) ABSTRACT

The invention relates to a non-human knockin animal having a mutation in a proBDNF gene wherein conversion from proBDNF to BDNF has been inhibited by the mutation.

### TECHNICAL FIELD

The present invention relates to a non-human knockin animal, particularly a non-human knockin animal in which a conversion from a precursor of BDNF (proBDNF) to a mature type (BDNF) by cleavage of protease in vivo has been inhibited/suppressed, a method for producing the animal, a vector for producing the animal as well as a method for screening medicaments used for abnormal behavior and nervous system diseases.

### BACKGROUND ART

In the nervous system, there are a “neurotrophic factor” which facilitates the existence of nerve cells and a “nerve cell death facilitating factor” which facilitates the death of nerve cells. A representative of the former is BDNF (Brain-derived neurotrophic factor), and the study on it has been advanced since 1990s (e.g., see Patent Document 1). However, the study on the latter has not been advanced.

Patent Document 1: JP Hei-05-328974 A

### DISCLOSURE OF INVENTION

### Problem to be Solved by the Invention

The present invention aims at providing a non-human knockin animal in which the nerve cell death facilitating factor has been introduced in order to elucidate functions of the nerve cell death facilitating factor.

The present invention also aims at providing a method for screening medicaments for treating diseases caused by the nerve cell death facilitating factor using the non-human knockin animal.

### MEANS FOR SOLVING THE PROBLEM

The present inventor has found that when a mutation is introduced in BDNF which is the neurotrophic factor, the conversion (maturation) from proBDNF to the mature type BDNF can be inhibited, and that the mutant proBDNF functions as the nerve cell death facilitating factor.

It has been found that the non-human knockin animal in which the mutant proBDNF whose conversion to the mature type BDNF had been inhibited was introduced exhibits characteristic abnormal behaviors such as stumbling and tumbling during walking and remarkably increased quantities of activity in an open field, and is useful as a model animal which causes the abnormal behaviors, mental diseases and nervous diseases.

The present invention relates to the following non-human knockin animal, vector for producing the animal, as well as the method for screening the medicaments.

[1] A non-human knockin animal having a mutation in proBDNF gene wherein conversion from proBDNF to BDNF has been inhibited by the mutation.

[2] The non-human knockin animal according to [1] wherein the proBDNF gene having the mutation is any of the following (a) to (c):

(a) DNA comprising a base sequence represented by SEQ ID NO:3 or a complementary chain thereto;

(b) DNA which hybridizes with the DNA comprising the base sequence represented by SEQ ID NO:3 under a stringent condition and encodes proBDNF in which the conversion to BDNF has been inhibited; and

(c) DNA encoding an amino acid sequence having one or more amino acid substitutions, additions, deletions or insertions in an amino acid sequence represented by SEQ ID NO:2 and in which the conversion from proBDNF to BDNF has been inhibited.

[3] The non-human knockin animal according to [1] wherein the animal is a mouse.

[4] The non-human knockin animal according to any of [1] to [3] wherein a position 125 and/or 127 in proBDNF gene has been mutated

[5] The non-human knockin animal according to [4] wherein the mutant proBDNF has mutations of R125M and R127L.

[6] A vector for making a non-human knockin animal having a mutant proBDNF gene in which conversion from proBDNF to BDNF has been inhibited.

[7] The vector according to [6] wherein the mutant proBDNF has been inserted in a position sandwiched with a long arm and a short arm.

[8] A method for screening medicaments for at least one disease selected from abnormal behaviors and mental diseases, characterized in that a medicament candidate substance is administered to the non-human knockin animal according to any of [1] to [5].

[9] The method according to [8] wherein the disease is at least one selected from the group consisting of diseases based on abnormality of cerebellum, vestibule or inner ear, diseases based on reduction of muscular force, temper tantrum, learning disability, ADHD (attention deficit/hyperactivity disorder), high-functioning autism, obsessive-compulsive disorder, schizophrenia, pervasive developmental disease, anorexia, dementia, sleep disturbance, panic disorder, neurosis, manic psychosis, depressive psychosis, bipolar disorder, psychosomatic disorder, anxiety disorders and intension.

[10] The method for screening according to [8] or [9], characterized in that an efficacy of a medicament candidate compound is determined using it as an indicator whether an expressed amount of at least one gene which increases or decreases 2 times or more relative to that in a wild type animal with mutation of proBDNF gene comes close to an expressed amount of the wild type or not by administering the medicament candidate compound.

### EFFECT OF THE INVENTION

The non-human knockin animal in which the gene of a proBDNF derivative which did not undergo the activation by processing had been introduced expressed characteristic phenotypes such as rolling when started to walk, staggering gait, thrashing with lying upside down and lowering of a lower body in an attitude. The animal also exhibited remarkable overactivity compared with wild type mice, and the quantity of activity in a dark phase (night), particularly for several hours after transition from a light phase to the dark phase was increased. Furthermore, an akinesia time period was significantly prolonged compared with the wild type mice in a tail suspension test, showing that the animal had mental abnormality of depression.

By observing the effects of various medicament candidate compounds on such phenotypes, it becomes possible to screen various therapeutic drugs for various diseases such as diseases based on abnormality of cerebellum, vestibule or inner ear, diseases based on reduction of muscular force, temper tantrum, motor function disorder, obsessive-compulsive disorder, schizophrenia, pervasive developmental disease (PDD), anorexia, dementia, sleep disturbance, panic disorder, neurosis, manic psychosis, depressive psychosis (including depression state, depression neurosis, hypobulia), bipolar disorder, psychosomatic disorder, anxiety disorders and intension.

In addition, the non-human knockin animal of the present invention not only easily takes a tumble by loosing sense of balance but also exhibits a remarkably higher quantity of motion than the wild type animal along with growth and a restless behavior, and is useful as a quite new model animal for LD (learning disability), ADHD (attention deficit/hyperactivity disorder), high-functioning autism, diseases based on abnormality of cerebellum, vestibule or inner ear, diseases based on reduction of muscular force, temper tantrum, motor function disorder, obsessive-compulsive disorder, schizophrenia, pervasive developmental disease (PDD), anorexia, dementia, sleep disturbance, panic disorder, neurosis, manic psychosis, depressive psychosis, bipolar disorder, psychosomatic disorder, anxiety disorders and intension.

### BRIEF DESCRIPTION OF THE DRAWINGS

FIG. 1 is a schematic view of a targeting vector;

FIG. 2 shows an amino acid sequence of proBDNF (entire sequence) and mature BDNF (underlined). A downward arrow indicates a cleaved site;

FIG. 3 shows a photograph of a mutant BDNF gene-knockin mouse and changes of body weight. (A) A wild type mouse (right) and the mutant BDNF gene-knockin homozygous mouse in littermates aged 5 weeks. (B) The mutant mice gain the body weight and grow with weeks of age. WT: wild type, ht: heterozygote and hm: homozygote;

FIG. 4 shows comparison of activity quantity for 5 minutes in an open field. An activity pattern for 5 minutes in the circular field was traced. The activity quantity in the mutant mouse (Mutant) was more remarkable compared with the wild type mouse (Wild);

FIG. 5 shows behavioral patterns for 25 minutes in the open field. An activity distance for 5 minutes in the circular field was plotted for 4 wild type mice (Wild) and 4 mutant mice (Mutant). The wild type mice were adapted to a novel environment and gradually shortened their behavioral distance. However, the mutant mice did not show such an adaptability and continued the active state;

FIG. 6 shows an inhibitory effect of an anxiolytic drug (etizolam) on abnormal behavior of the mutant BDNF gene-knockin mice. P: administration of PBS; E: administration of etizolam, A test for significant difference was performed by t-test;

FIG. 7 shows results of measuring a locomotor activity in a home cage for about 5 days using the mutant BDNF gene-knockin mice (Mutant) and the wild type mice (Wild). A dark phase (12 hours) and a light phase (12 hours) are represented by black and white, respectively in a horizontal axis. The locomotor activity quantity is remarkable in the knockin mice (Mutant);

FIG. 8 shows abnormality in cerebellum morphology of the mutant BDNF gene-knockin mouse. A groove (upper arrow) between VIb lobe and VII and a groove (left arrow) of IX lobe disappeared in the mutant BDNF gene-knockin homozygous mouse (Homo). However, the grooves were present in the mutant BDNF gene-knockin heterozygous mouse (Hetero) and the wild type mouse (Wild);

FIG. 9 is a view showing electrophoresis patterns of genotyping; and

FIG. 10 shows the results of a tail suspension test for measuring a depressive state in the mutant BDNF gene-knockin mice. (A) Transition of representative akinesia time period in the tail suspension test. Wild type mouse (gray circle) and mutant BDNF gene-knockin homozygous mouse (black circle) aged 6 to 7 weeks. Immediately after the mouse was suspended using a tail suspension apparatus, the measurement was started and the akinesia time period was measured for 6 minutes. A vertical axis represents the akinesia time period in each session. (B) A graph of akinesia time period for 6 minutes in 8 mice having each genotype (aged 6 to 7 weeks), p<0.0001 (t-test). The akinesia time period in the mutant mice is much longer than that in the wild type mice, indicating that the mutant mice take a depressive behavior.

### BEST MODE FOR CARRYING OUT THE INVENTION

In the present invention, the sequence of a naturally occurring type of human proBDNF protein is represented by SEQ ID NO:1, and the sequence of a non-cleavable proBDNF derivative protein (hereinafter sometimes abbreviated as “proBDNF-ML”) in which Arg at position 125 has been substituted with Net (R125M) and Arg at position 127 has been substituted with Leu (R127L) is represented by SEQ ID NO:2. A gene corresponding to SEQ ID NO:2 is represented by SEQ ID NO:3.

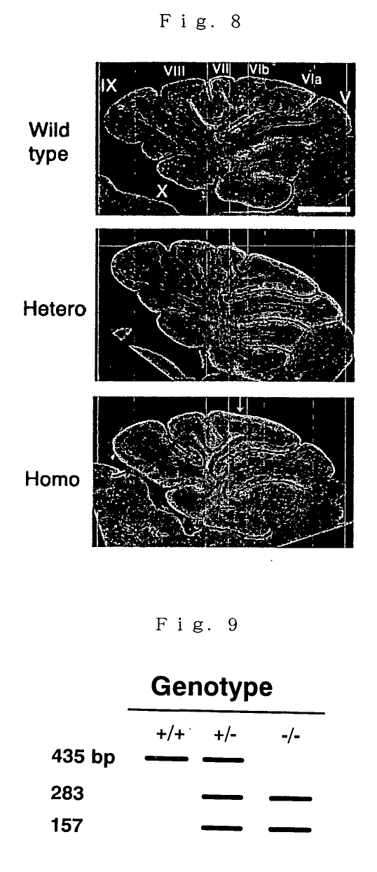

It has been revealed that the non-cleavable proBDNF derivative (BDNF-ML) represented by SEQ ID NO:2 has harmful actions upon nerve cells, e.g., facilitation of dropout of fibers in cholinergic nerve cells and facilitation of cell death of cerebellar granular nerve cells. For example, as shown in FIG. 8, in the cerebellum in a mutant proBDNF knockin mouse, a groove between VIb lobe and VII and a groove of IX lobe disappear. The characteristic phenotypes in the non-human knockin animals of the present invention are likely based on such actions of the mutant proBDNF.

The mutant proBDNF used for making the non-human knockin animals of the present invention includes:

(a) DNA comprising a base sequence represented by SEQ ID NO:3 or a complementary chain thereof;

(b) DNA which hybridizes with the DNA comprising the base sequence represented by SEQ ID NO:3 under the stringent condition and encodes proBDNF whose conversion to BDNF has been inhibited; and

(c) DNA which encodes an amino acid sequence having one or more amino acid substitutions, additions, deletions or insertions in an amino acid sequence represented by SEQ ID NO:2, and in which the conversion from proBDNF to BDNF has been inhibited.

The above substitution, addition, deletion or insertion of amino acids in the amino acid sequence can be performed by gene engineering techniques such as site-specific mutagenesis (Methods in Enzymology, 154, 350, 367-382 (1987); ibid., 100, 468 (1983); Nucleic Acids Res., 12, 9441 (1984); Zoku Seikagaku Jikken Kouza I “Idenshi Kenkyuho II” p. 105, 1986 edited by the Japanese Biochemical Society), chemical synthesis means such as phosphate triester method and phosphate amidite method (J. Am. Chem. Soc., 89, 4801(1967); ibid., 91, 3350 (1969); Science, 150, 178 (1968); Tetrahedron Lett., 22, 1859 (1981); ibid., 24, 245 (1983)) and combinations thereof. As the number of amino acids substituted, added, deleted or inserted, one to multiple amino acids, preferably one to over ten amino acids, and more preferably one to several amino acids are preferably exemplified for expressing the phenotype in the non-human knockin animals of the present invention. The protein of SEQ ID NO:2 includes a signal peptide, and the signal peptide can be further deleted.

The stringent condition includes the ordinary condition used for primers and probes, is not particularly limited, and, for example, the condition in 0.2×SSC containing 0.1% SDS at 50° C. or the condition in 1×SSC containing 0.1% SDS at 60° C. can be exemplified.

An origin of the naturally occurring type of proBDNF is not particularly limited, and it is possible to widely exemplify animals such as mammalian animals such as human beings, cattle, swines, monkeys, dogs, sheeps, goats, rabbits, mice and rats, birds such as chickens and ducks, amphibians such as frogs and reptiles such as efts. The naturally occurring type of proBDNF can be used as a production material of the mutant proBDNF whose conversion to the mature type BDNF (by protease cleavage) has been inhibited.

Production of the non-human knockin animal of the present invention is not particularly limited, and can be performed by homologous recombination using a gene construct or the vector.

The method for producing the non-human knockin animal using the vector is exemplified below.

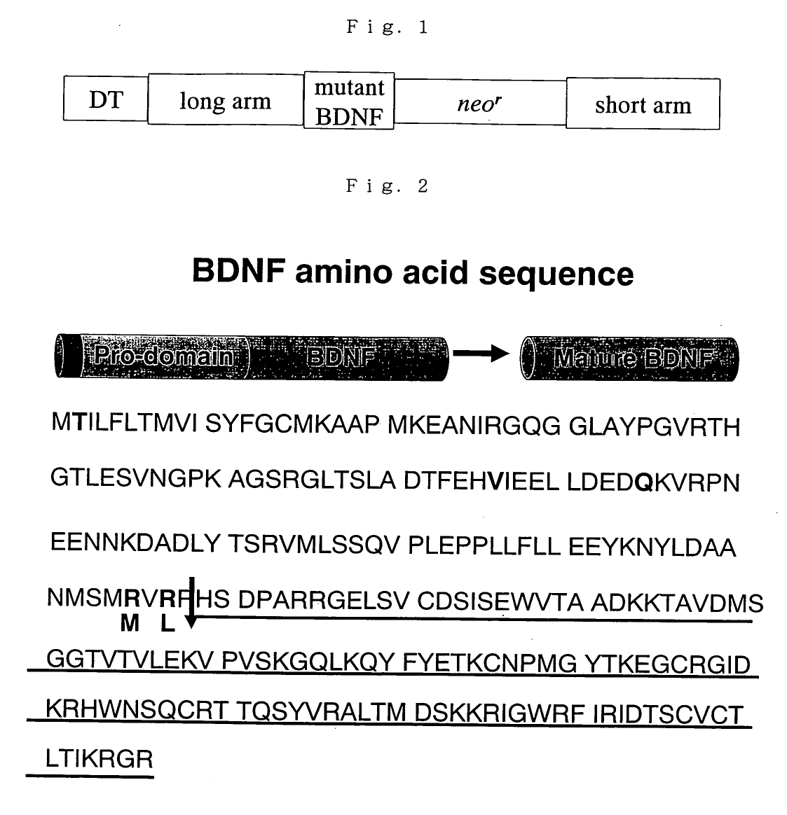

In preferable embodiments of the present invention, the vector for producing the non-human knockin animal has a long arm and a short arm for the homologous recombination, and a marker for selecting recombinants as shown in FIG. 1.

The mutant proBDNF gene used in the present invention can be produced using the naturally occurring type proBDNF gene known publicly and appropriate primers according to standard methods. The resulting mutant proBDNF gene is introduced between the long art and the short arm in FIG. 1. At that time, by using a drug resistant gene such as neomycin resistant gene (neo^{r}), it is possible to select the animal in which the gene has been introduced (negative selection). The marker gene for the selection may be the drug resistant gene alone, but by further linking a diphtheria toxin (DT) gene to perform the positive selection, it is possible to more easily perform the selection.

Since the wild type animal has a [long arm]-[proBDNF]-[short arm] structure, by performing the homologous recombination in ES cells using the vector in FIG. 1, it is possible to obtain the non-human knockin animal of the present invention, in which the mutant proBDNF has been introduced.

A tag such as myc can also be attached to the mutant proBDNF. For example, the localization of proBDNF at a tissue level can be analyzed by a myc tag.

In the non-human knockin animals of the present invention, both a heterozygote and a homozygote as the genotype are useful. The homozygous animal exhibits the characteristics such as easily taking a tumble, hyperkinesis, restless, low body weight and small size strongly, and the heterozygous animal has intermediate phenotypes. This suggests that these phenotypes are closely associated with the non-cleavable proBDNF.

The non-human animals include mammalian animals such as mice, rats, hamsters, rabbits, dogs and monkeys, or reptiles and amphibians.

The drug resistant gene (marker) such as Neo may be removed by crossing with Cre mice (B6.Cg-Tg(CAG-cre)CZ-MO2Osb).

Furthermore, gene expression levels were measured using DNA arrays for the non-human knockin mice in which the mutant proBDNF had been introduced and the wild type mice. The results are shown in Table 1 where the expressed amount was reduced to 0.5 times or less relative to that in the wild type mouse, Table 2 where the amount was increased to 2.0 times or more, Table 3 where the amount was reduced to 0.5 times or less and the expression amount was large and Table 4 where the amount was increased to 2.0 times or more and the expression amount was large.

TABLE 1

| Genbank No. | Gene Title |
|---|---|
| BB311104 | dendritic cell protein GA17 |
| NM_053181 | expressed sequence AA415817 |
| AF373288 | a disintegrin and metalloprotease domain 34 |
| AV009804 | AV009804 Mus musculus 18-day embryo C57BL/6J Mus musculus cDNA clone |
| | 1110020N03, mRNA sequence. |
| AI428125 | RIKEN cDNA 4921511M17 gene |
| BE980528 | Clone IMAGE: 2647821, mRNA |
| BM227718 | hypothetical protein 4931417A20 |
| BG065575 | Transcribed sequences |
| AF102134 | CD22 antigen |
| AK015547 | Mus musculus adult male testis cDNA, RIKEN full-length enriched library, |
| | clone: 4930471E07 product: unclassifiable, full insert sequence. |
| BB362079 | RIKEN cDNA C130009A20 gene |
| BG095528 | uu88e10.x1 Soares_mouse_NMGB_bcell Mus musculus cDNA clone IMAGE: 3383539 |
| | 3′ similar to TR: Q9Y3X8 Q9Y3X8 HYPOTHETICAL 60.5 KD PROTEIN;, mRNA |
| | sequence. |
| AY057913 | brain derived neurotrophic factor |
| BB306259 | Adult male corpora quadrigemina cDNA, RIKEN full-length enriched library, |
| | clone: B230207H05 product: unknown EST, full insert sequence |
| BG068520 | Transcribed sequences |
| BB462504 | 12 days embryo spinal ganglion cDNA, RIKEN full-length enriched library, |
| | clone: D130076J23 product: unknown EST, full insert sequence |
| BB426900 | hypothetical protein C630004L07 |
| AV007708 | AV007708 Mus musculus 18-day embryo C57BL/6J Mus musculus cDNA clone |
| | 1110005N20, mRNA sequence. |
| BC010747 | cytochrome P450, family 4, subfamily a, polypeptide 10 |
| BB189091 | Transcribed sequences |
| NM_009873 | cyclin-dependent kinase 6 |
| BG069348 | H3076D04-3 NIA Mouse 15K cDNA Clone Set Mus musculus cDNA clone |
| | H3076D04 3′, mRNA sequence. |
| AV215734 | AV215734 RIKEN full-length enriched, ES cells Mus musculus cDNA clone |
| | 2410152E03 3′, mRNA sequence. |
| BG066203 | Transcribed sequences |
| BM237858 | programmed cell death 10 |
| AK020392 | Adult male diencephalon cDNA, RIKEN full-length enriched library, |
| | clone: 9330185C12 product: unclassifiable, full insert sequence |
| BM227750 | Similar to preferentially expressed antigen in melanoma like 4 (LOC386474), mRNA |
| BB713538 | cDNA sequence BC013667 |
| BI319366 | ie43c12.x1 Kaestner ngn3 wt Mus musculus cDNA 3′ similar to TR: O55080 O55080 |
| | NON-SELECTIVE CATION CHANNEL.;, mRNA sequence. |
| NM_009042 | regenerating islet-derived 1 |
| AW990746 | cDNA sequence BC038613 |
| NM_138674 | polycystic kidney and hepatic disease 1-like 1 |
| NM_010983 | olfactory receptor 2 |
| AV266902 | Adult male testis cDNA, RIKEN full-length enriched library, clone: 4930521G14 |
| | product: unknown EST, full insert sequence |
| BC024118 | RIKEN cDNA 9430059P22 gene |
| BB104484 | Transcribed sequences |
| BB084182 | BB084182 RIKEN full-length enriched, adult male diencephalon Mus musculus |
| | cDNA clone 9330187H22 3′, mRNA sequence. |
| BB417508 | archease |
| BG066329 | Transcribed sequences |
| BG069192 | Transcribed sequences |
| NM_015811 | regulator of G-protein signaling 1 |
| U80889 | CAG trinucleotide repeat mRNA, partial sequence |
| BB294794 | Transcribed sequences |
| AU045440 | Transcribed sequences |
| NM_008350 | interleukin 11 |
| BB080402 | Transcribed sequences |
| NM_011093 | paired-Ig-like receptor A6 |
| BB105009 | 12 days embryo embryonic body between diaphragm region and neck cDNA, RIKEN |
| | full-length enriched library, clone: 9430093N11 product: unknown EST, full insert |
| | sequence |
| BB806657 | Transcribed sequences |
| AK019686 | peptidylprolyl isomerase F, opposite strand transcription unit |
| C78858 | C78858 Mouse 3.5-dpc blastocyst cDNA Mus musculus cDNA clone J0056D12 3′, |
| | mRNA sequence. |
| C77984 | Transcribed sequences |
| BB021606 | Transcribed sequences |
| AI504626 | CDNA clone IMAGE: 4024374, with apparent retained intron |
| BG229036 | Transcribed sequence with weak similarity to protein ref: NP_081764.1 |
| | (M. musculus) RIKEN cDNA 5730493819 [Mus musculus] |
| NM_020568 | plasma membrane associated protein, S3-12 |
| AK009671 | RIKEN cDNA 2310038E17 gene |
| AK020543 | Mus musculus adult male urinary bladder cDNA, RIKEN full-length enriched library, |
| | clone: 9530004M16 product: hypothetical protein, full insert sequence. |
| BC006937 | asrij protein |
| BB468375 | Transcribed sequences |
| AI596396 | RIKEN cDNA 6330500C13 gene |
| AA517747 | vh80h04.r1 Knowles Solter mouse E6 5d whole embryo Mus musculus cDNA clone |
| | IMAGE: 893335 3′, mRNA sequence. |
| NM_033314 | solute carrier organic anion transporter family, member 2a1 |
| BE457051 | RIKEN cDNA 4933425I22 gene |
| AK014988 | suppressor of cytokine signaling 4 |
| AK006825 | RIKEN cDNA 1700057K13 gene |
| AV347405 | pleckstrin homology, Sec7 and coiled-coil domains 1 |
| AK020714 | cytochrome P450, family 4, subfamily f, polypeptide 18 |
| AV367395 | cDNA sequence BC038479 |
| BF319562 | RIKEN cDNA 4933432N21 gene |
| C80272 | nuclear receptor subfamily 5, group A, member 2 |
| AK014028 | 13 days embryo head cDNA, RIKEN full-length enriched library, clone: 3110030C24 |
| | product: unclassifiable, full insert sequence |
| BE310730 | cyclin G associated kinase |
| BB040523 | BB040523 RIKEN full-length enriched, 13 days embryo male testis Mus musculus |
| | cDNA clone 6030449L13 3′ similar to D38616 Human mRNA for phosphorylase |
| | kinase alpha subunit, mRNA sequence. |
| AV247312 | Transcribed sequences |
| AW822930 | Similar to PITPNM family member 3; PYK2 N-terminal domain-interacting receptor |
| | 1; retinal degeneration B alpha 3 (Drosophila) (LOC216884), mRNA |
| U80892 | Mus musculus CAG trinucleotide repeat mRNA, partial sequence. |
| AK015606 | RIKEN cDNA 4930481F22 gene |
| BC021322 | glycogen synthase 2 |
| BB249544 | Transcribed sequences |
| AV090279 | Adult male tongue cDNA, RIKEN full-length enriched library, clone: 2310047N11 |
| | product: unknown EST, full insert sequence |
| AF425084 | serine (or cysteine) proteinase inhibitor, clade B, member 6c |
| BB597421 | RIKEN cDNA 3110035P10 gene |
| BB321076 | RIKEN cDNA B230396O12 gene |
| AF361350 | calcium channel, voltage-dependent, gamma subunit 8 |
| BB462381 | Transcribed sequences |
| AV025588 | gelsolin |
| AK018356 | 12 days embryo female mullerian duct includes surrounding region cDNA, RIKEN |
| | full-length enriched library, clone: 6820402A03 product: unclassifiable, full insert |
| | sequence |
| BB225339 | RIKEN cDNA 2610034K17 gene |
| R75193 | MDB1137 Mouse brain, Stratagene Mus musculus cDNA 3′end, mRNA sequence. |
| AK018007 | RIKEN cDNA 5830453K13 gene |
| AW495649 | RIKEN cDNA 4930422I07 gene |
| AK016421 | RIKEN cDNA 4931400O07 gene |
| BB420672 | Transcribed sequence with weak similarity to protein pir: S12207 (M. musculus) |
| | S12207 hypothetical protein |
| NM_133709 | chordin-like 2 |
| BB496620 | Transcribed sequence with weak similarity to protein sp: P20618 (H. sapiens) |
| | PSB1_HUMAN Proteasome subunit beta type 1 |
| AK012858 | septin 6 |
| BB049112 | BB049112 RIKEN full-length enriched, adult male cerebellum Mus musculus cDNA |
| | clone 6530403B10 3′, mRNA sequence. |
| BE979553 | Transcribed sequences |
| AK015277 | unnamed protein product; putative weakly similar to COMPLEMENT FACTOR H- |
| | RELATED PROTEIN 1 PRECURSOR (FHR-1) (H FACTOR-LIKE PROTEIN 1) (H- |
| | FACTOR LIKE 1) (H36) [Homo sapiens] (SWISSPROT\|Q03591, evidence: FASTY, |
| | 51% ID, 75.1% length, match = 723); Mus musculus adult male testis cDNA, RIKEN full- |
| | length enriched library, clone: 4930431N10 product: weakly similar to COMPLEMENT |
| | FACTOR H-RELATED PROTEIN 1 PRECURSOR (FHR-1) (H FACTOR-LIKE |
| | PROTEIN 1) (H-FACTOR LIKE 1) (H36) [Homo sapiens], full insert sequence. |
| NM_009426 | thyrotropin releasing hormone |
| BB440892 | 9 days embryo whole body cDNA, RIKEN full-length enriched library, |
| | clone: D030024E12 product: unknown EST, full insert sequence |
| C77631 | C77631 Mouse 3.5-dpc blastocyst cDNA Mus musculus cDNA clone J0035A08 3′ |
| | similar to Mouse T-cell receptor (TCR V-alpha 16.1) gene exons 1-2, mRNA, |
| | mRNA sequence. |
| BB436898 | BB436898 RIKEN full-length enriched, adult pancreas Islet cells Mus musculus |
| | cDNA clone C820020L21 3′, mRNA sequence. |
| BM248709 | RIKEN cDNA 9530008N10 gene |
| AW048864 | cholinergic receptor, nicotinic, alpha polypeptide 6 |
| BC027121 | RIKEN cDNA 2600017H08 gene |
| BG065704 | Transcribed sequences |
| BB393897 | 2 days neonate thymus thymic cells cDNA, RIKEN full-length enriched library, |
| | clone: E430005M11 product: unknown EST, full insert sequence |
| BF465041 | UI-M-CG0p-bqg-a-12-0-UI.s1 NIH_BMAP_Ret4_S2 Mus musculus cDNA clone UI- |
| | M-CG0p-bqg-a-12-0-UI 3′, mRNA sequence. |
| NM_025684 | RIKEN cDNA 5730521E12 gene |
| BB120697 | Transcribed sequence with weak similarity to protein prf: 2113200B (H. sapiens) |
| | 2113200B ribosomal protein L21 [Homo sapiens] |
| AU021836 | Transcribed sequences |
| BB649821 | Transcribed sequences |
| BB455609 | 12 days embryo spinal ganglion cDNA, RIKEN full-length enriched library, |
| | clone: D130039D18 product: unknown EST, full insert sequence |
| BC019726 | polyadenylate binding protein-interacting protein 1 |
| BB043424 | a disintegrin-like and metalloprotease (reprolysin type) with thrombospondin type 1 |
| | motif, 5 (aggrecanase-2) |
| BB824671 | BB824671 RIKEN full-length enriched, mammary gland RCB-0526 Jyg-MC(A) |
| | cDNA Mus musculus cDNA clone G830034D04 3′, mRNA sequence. |
| BG069640 | Transcribed sequences |
| AW123150 | Transcribed sequences |
| BG087177 | RIKEN cDNA D130062J10 gene |
| NM_008199 | histocompatibility 2, blastocyst |
| BB453954 | 12 days embryo spinal ganglion cDNA, RIKEN full-length enriched library, |
| | clone: D130027L03 product: unknown EST, full insert sequence |
| AU080871 | AU080871 Sugano mouse brain mncb Mus musculus cDNA clone MNCb-6175 5′, |
| | mRNA sequence. |
| BB012080 | BB012080 RIKEN full-length enriched, adult male testis (DH10B) Mus musculus |
| | cDNA clone 4930407C07 3′ similar to X60785 Cricetulus griseus (chinese hamster) |
| | mRNA for beta tubulin (clone B3T), mRNA sequence. |
| BB138185 | latent transforming growth factor beta binding protein 1 |
| NM_008880 | phospholipid scramblase 2 |
| NM_009637 | AE binding protein 2 |
| NM_130890 | calpain 8 |
| AV094878 | expressed sequence AI449441 |
| NM_011355 | SFFV proviral integration 1 |
| NM_021554 | RIKEN cDNA 0610012D09 gene |
| BQ266161 | calcium channel, voltage-dependent, gamma subunit 8 |
| BE947490 | Transcribed sequences |
| BB076877 | Transcribed sequences |
| BB168878 | BB168878 RIKEN full-length enriched, adult male hypothalamus Mus musculus |
| | cDNA clone A230002G06 3′ similar to U41060 Human breast cancer, estrogen |
| | regulated LIV-1 protein (LIV-1) mRNA, mRNA sequence. |
| BB297502 | G protein-coupled receptor 23 |
| BM237637 | Transcribed sequence with weak similarity to protein ref: NP_081764.1 |
| | (M. musculus) RIKEN cDNA 5730493B19 [Mus musculus] |
| BG068277 | Transcribed sequences |
| BG078968 | |
| BB547375 | POU domain, class 4, transcription factor 2 |
| BB283617 | COP9 (constitutive photomorphogenic) homolog, subunit 3 (Arabidopsis thaliana) |
| BB448877 | expressed sequence AI256775 |
| AK016763 | Rho GTPase activating protein 19 |
| BB800086 | 3-monooxgenase/tryptophan 5-monooxgenase activation protein, gamma |
| | polypeptide |
| AU022077 | I(3)mbt-like 3 (Drosophila) |
| BB483579 | general transcription factor II E, polypeptide 1 (alpha subunit) |
| AV328280 | solute carrier family 1 (glial high affinity glutamate transporter), member 2 |
| BB010597 | DNA methyltransferase 2 |
| BB755358 | mitochondrial carrier homolog 2 (C. elegans) |
| BM939280 | UI-M-BZ1-bkm-e-10-0-UI.r1 NIH_BMAP_MHI2_S1 Mus musculus cDNA clone UI- |
| | M-BZ1-bkm-e-10-0-UI 5′, mRNA sequence. |
| BB503481 | BB503481 RIKEN full-length enriched, 0 day neonate kidney Mus musculus cDNA |
| | clone D630043I17 3′, mRNA sequence. |
| BB450318 | BB450318 RIKEN full-length enriched, 12 days embryo spinal ganglion Mus |
| | musculus cDNA clone D130002A10 3′, mRNA sequence. |
| AA165749 | RIKEN cDNA 3110006P09 gene |
| AK017798 | GULP, engulfment adaptor PTB domain containing 1 |
| NM_021892 | RFamide-related peptide |
| AV209518 | AV209518 RIKEN full-length enriched, adult male testis Mus musculus cDNA clone |
| | 1700120A01 3′, mRNA sequence. |
| BB107526 | synaptopodin 2 |
| BM239026 | 12 days embryo embryonic body between diaphragm region and neck cDNA, RIKEN |
| | full-length enriched library, clone: 9430088I02 product: unknown EST, full insert |
| | sequence |
| AU040128 | Transcribed sequences |
| AW492576 | Transcribed sequences |
| NM_019952 | B-cell stimulating factor 3 |
| AU019880 | AU019880 Mouse eight-cell stage embryo cDNA Mus musculus cDNA clone |
| | J0523G12 3′, mRNA sequence. |
| BB043407 | BB043407 RIKEN full-length enriched, 13 days embryo male testis Mus musculus |
| | cDNA clone 6030473G23 3′, mRNA sequence. |
| BC002159 | RIKEN cDNA 4632408A20 gene |
| NM_030266 | inositol polyphosphate-4-phosphatase, type I |
| AI462524 | serine (or cysteine) proteinase inhibitor, clade B, member 5 |
| NM_011376 | single-minded 1 |
| BG068380 | Transcribed sequences |
| AK011311 | RIKEN cDNA 2610005B21 gene |
| BI081061 | cell division cycle associated 3 |
| BB104484 | Transcribed sequences |
| BC016221 | kinesin family member 1C |
| AI850249 | Transcribed sequences |
| BM507022 | Transcribed sequence with weak similarity to protein pir: I58401 (M. musculus) |
| | I58401 protein-tyrosine kinase |
| AK005507 | CD59a antigen |
| BI692117 | angiomotin-like 1 |
| X57938 | POU domain, class 2, transcription factor 2 |
| AV353171 | 11 days embryo gonad cDNA, RIKEN full-length enriched library, clone: 7030422C13 |
| | product: unclassifiable, full insert sequence |
| BB091390 | testis specific gene A14 |
| AK016015 | cadherin 23 (otocadherin) |
| BF302166 | guanine nucleotide binding protein, alpha 12 |
| AK016178 | Mus musculus adult male testis cDNA, RIKEN full-length enriched library, |
| | clone: 4930558J22 product: histone 4 protein, full insert sequence. |
| NM_134069 | solute carrier family 17 (sodium phosphate), member 3 |
| NM_009534 | yes-associated protein |
| AV259535 | RIKEN cDNA 1700007K09 gene |
| AK017963 | Adult male thymus cDNA, RIKEN full-length enriched library, clone: 5830432F11 |
| | product: unclassifiable, full insert sequence |
| AK018213 | Mus musculus adult male medulla oblongata cDNA, RIKEN full-length enriched |
| | library, clone: 6330514J04 product: unknown EST, full insert sequence. |
| BB767775 | naked cuticle 2 homolog (Drosophila) |
| AK016492 | Adult male testis cDNA, RIKEN full-length enriched library, clone: 4931430N09 |
| | product: unclassifiable, full insert sequence |
| AK008016 | RIKEN cDNA 2010001M09 gene |
| BC021887 | paraoxonase 2 |
| BC023358 | RIKEN cDNA 0610009A07 gene |
| BB425852 | 12 days embryo spinal cord cDNA, RIKEN full-length enriched library, |
| | clone: C530049D23 product: unknown EST, full insert sequence |
| BI412259 | RIKEN cDNA E130112L23 gene |
| BB535350 | 0 day neonate lung cDNA, RIKEN full-length enriched library, clone: E030042P18 |
| | product: unclassifiable, full insert sequence |
| AK013705 | RIKEN cDNA 2900056M07 gene |
| AK014864 | Adult male testis cDNA, RIKEN full-length enriched library, clone: 4921511C10 |
| | product: unclassifiable, full insert sequence |
| AA880220 | jagged 1 |
| AA672901 | vn70c04.r1 Barstead mouse irradiated colon MPLRB7 Mus musculus cDNA clone |
| | IMAGE: 1026534 5′, mRNA sequence. |
| BC006623 | RIKEN cDNA 1810046I24 gene |
| BB377300 | 16 days embryo head cDNA, RIKEN full-length enriched library, clone: C130088M18 |
| | product: unknown EST, full insert sequence |
| BG066582 | Transcribed sequence with strong similarity to protein pir: S12207 (M. musculus) |
| | S12207 hypothetical protein |
| C79445 | 18-day embryo whole body cDNA, RIKEN full-length enriched library, |
| | clone: 1100001P14 product: TUBULIN, BETA 5 homolog [Homo sapiens], full insert |
| | sequence |
| BI134670 | RIKEN cDNA 2410004024 gene |
| NM_080557 | sorting nexin 4 |
| AV291985 | Transcribed sequences |
| BB041089 | proprotein convertase subtilisin/kexin type 5 |
| BB824003 | gem (nuclear organelle) associated protein 5 |
| BC022133 | cDNA sequence BC022133 |
| AK008898 | Adult male stomach cDNA, RIKEN full-length enriched library, clone: 2210411G17 |
| | product: unclassifiable, full insert sequence |
| BM115255 | Transcribed sequences |
| BE946363 | adenylate cyclase 5 |
| BM293428 | nicastrin |
| AI481991 | vi22c10.x1 Barstead mouse proximal colon MPLRB6 Mus musculus cDNA clone |
| | IMAGE: 904530 3′ similar to gb: K00129 Mouse MHC class I H2-D gene (MOUSE);, |
| | mRNA sequence. |
| AV250628 | AV250628 RIKEN full-length enriched, 0 day neonate head Mus musculus cDNA |
| | clone 4833424P18 3′, mRNA sequence. |
| BE988930 | Similar to dJ259A10.1 (ssDNA binding protein (SEB4D)) (LOC380843), mRNA |
| NM_022033 | 3-oxoacid CoA transferase 2A |
| BB100157 | BB100157 RIKEN full-length enriched, 12 days embryo, embryonic body between |
| | diaphragm region and neck Mus musculus cDNA clone 9430071J20 3′, mRNA |
| | sequence. |
| AV338420 | AV338420 RIKEN full-length enriched, adult male olfactory bulb Mus musculus |
| | cDNA clone 6430409E08 3′, mRNA sequence. |
| BB050456 | 12 days embryo male wolffian duct includes surrounding region cDNA, RIKEN full- |
| | length enriched library, clone: 6720406O06 product: unknown EST, full insert |
| | sequence |
| AK005886 | RIKEN cDNA 1700012A16 gene |
| BB124626 | BB124626 RIKEN full-length enriched, adult male urinary bladder Mus musculus |
| | cDNA clone 9530098J09 3′, mRNA sequence. |
| AI507229 | deoxyribonuclease 1-like 2 |
| AJ249392 | RIKEN cDNA 1700021K02 gene |
| NM_026204 | RIKEN cDNA 1700001G17 gene |
| BB775785 | paired immunoglobin-like type 2 receptor alpha |
| BG072108 | expressed sequence AI449705 |
| BB014981 | RIKEN cDNA C330046L10 gene |
| AJ002522 | myosin, heavy polypeptide 1, skeletal muscle, adult |
| BB485245 | RIKEN cDNA E130115J16 gene |
| BB709811 | cDNA sequence BC031748 |
| AK016849 | Adult male testis cDNA, RIKEN full-length enriched library, clone: 4933417N07 |
| | product: unknown EST, full insert sequence |
| BC027227 | Clone IMAGE: 2609806, mRNA |
| AI790773 | cytochrome P450, family 2, subfamily j, polypeptide 11 |
| BM119869 | Transcribed sequences |
| AV307358 | Transcribed sequences |
| AI448971 | Transcribed sequences |
| AV233462 | 0 day neonate skin cDNA, RIKEN full-length enriched library, clone: 4632424N07 |
| | product: unknown EST, full insert sequence |
| BB476639 | steroid 5 alpha-reductase 2-like 2 |
| BB386302 | Transcribed sequences |
| AK006008 | genetic suppressor element 1 |
| BB011474 | Transcribed sequence with weak similarity to protein sp: P51855 (M. musculus) |
| | GSHB_MOUSE Glutathione synthetase |
| C80425 | Transcribed sequence with moderate similarity to protein ref: NP_081764.1 |
| | (M. musculus) RIKEN cDNA 5730493B19 [Mus musculus] |
| BB457749 | 12 days embryo spinal ganglion cDNA, RIKEN full-length enriched library, |
| | clone: D130053H13 product: unknown EST, full insert sequence |
| NM_138685 | RIKEN cDNA 9230106L14 gene |
| AF288379 | killer cell lectin-like receptor, subfamily A, member 7 |
| BB461344 | doublesex and mab-3 related transcription factor like family A1 |
| BC003748 | syntrophin, basic 1 |
| BB764453 | RIKEN cDNA 2010109H09 gene |
| NM_021416 | |
| AU022059 | AU022059 Mouse unfertilized egg cDNA Mus musculus cDNA clone J0407G03 3′, |
| | mRNA sequence. |
| BB747588 | BB747588 RIKEN full-length enriched, adult male kidney Mus musculus cDNA |
| | clone F530204N04 3′, mRNA sequence. |
| AU040911 | AU040911 Mouse four-cell-embryo cDNA Mus musculus cDNA clone J0820F02 3′, |
| | mRNA sequence. |
| NM_011709 | whey acidic protein |
| AV174739 | RIKEN cDNA 2210018M03 gene |
| BB233495 | BB233495 RIKEN full-length enriched, 3 days neonate thymus Mus musculus |
| | cDNA clone A630043O08 3′, mRNA sequence. |
| BE290548 | procollagen, type XXIII, alpha 1 |
| AI425983 | RIKEN cDNA 4930417P05 gene |
| AK012779 | RIKEN cDNA 2410007P03 gene |
| BB795733 | Adult male medulla oblongata cDNA, RIKEN full-length enriched library, |
| | clone: 6330417G03 product: unknown EST, full insert sequence |
| BC011413 | MRNA similar to ribosomal protein S20 (cDNA clone MGC: 6876 IMAGE: 2651405), |
| | complete cds |
| BQ031470 | Transcribed sequences |
| AK009753 | Mus musculus adult male tongue cDNA, RIKEN full-length enriched library, |
| | clone: 2310042I22 product: unknown EST, full insert sequence. |
| BF463689 | Transcribed sequences |
| BC025424 | zinc finger protein 106 |
| AK007309 | RIKEN cDNA 1700128E19 gene |
| BB735036 | Transcribed sequences |
| NM_008650 | methylmalonyl-Coenzyme A mutase |
| AV032530 | Transcribed sequence with strong similarity to protein sp: P00722 (E. coli) |
| | BGAL_ECOLI Beta-galactosidase |
| AV305197 | 2 days neonate thymus thymic cells cDNA, RIKEN full-length enriched library, |
| | clone: E430016M23 product: RIKEN cDNA 5730526G10 gene cluster member, full |
| | insert sequence |
| NM_026202 | RIKEN cDNA 2610529H08 gene |
| BB226761 | Adult male cecum cDNA, RIKEN full-length enriched library, clone: 9130413E14 |
| | product: unknown EST, full insert sequence |
| AF371320 | brain and acute leukemia, cytoplasmic |
| AK016516 | RIKEN cDNA 4931407K02 gene |
| BC021497 | RIKEN cDNA 5730444A13 gene |
| AK015625 | Mus musculus adult male testis cDNA, RIKEN full-length enriched library, |
| | clone: 4930485E13 product: unclassifiable, full insert sequence. |
| N0M_021358 | 5-hydroxytryptamine (serotonin) receptor 6 |
| BB656515 | RIKEN cDNA B430219L04 gene |
| AK019581 | nicotinamide nucleotide transhydrogenase |
| AW124625 | Transcribed sequences |
| BM935317 | Transcribed sequences |
| AK008821 | RIKEN cDNA 2210404A22 gene |
| AW490245 | Transcribed sequences |
| BM213278 | ES cells cDNA, RIKEN full-length enriched library, clone: 2410046H18 |
| | product: unknown EST, full insert sequence |
| AK018659 | 3 days neonate thymus cDNA, RIKEN full-length enriched library, |
| | clone: A630088H07 product: unknown EST, full insert sequence |
| AK018265 | cDNA sequence BC013565 |
| AB053307 | amyotrophic lateral sclerosis 2 (juvenile) homolog (human) |
| BB133021 | Transcribed sequence with moderate similarity to protein ref: NP_085911.1 |
| | (H. sapiens) DEAD/H |
| BE134115 | RIKEN cDNA C330005L02 gene |
| AK021221 | RIKEN cDNA C330024D12 gene |
| BB379753 | BB379753 RIKEN full-length enriched, 0 day neonate cerebellum Mus musculus |
| | cDNA clone C230001I10 3′, mRNA sequence. |
| BB335489 | matrix metalloproteinase 24 |
| NM_023066 | aspartate-beta-hydroxylase |
| NM_007819 | motile sperm domain containing 3 |
| AI882525 | CD47 antigen (Rh-related antigen, integrin-associated signal transducer) |
| BB533836 | BB533836 RIKEN full-length enriched, 0 day neonate lung Mus musculus cDNA |
| | clone E030032D12 3′, mRNA sequence. |
| NM_024199 | cleavage stimulation factor, 3′ pre-RNA, subunit 1 |
| BC003880 | motile sperm domain containing 3 |
| BB549552 | RIKEN cDNA 5730456K23 gene |
| BB333077 | BB333077 RIKEN full-length enriched, 10 days neonate medulla oblongata Mus |
| | musculus cDNA clone B830006O16 3′, mRNA sequence. |
| NM_008954 | persephin |
| AI414902 | mb56g02.x1 Soares mouse p3NMF19.5 Mus musculus cDNA clone IMAGE: 333458 |
| | 3′, mRNA sequence. |
| AW541149 | Transcribed sequences |
| AK012131 | RIKEN cDNA 2610511O17 gene |
| AK017045 | Mus musculus adult male testis cDNA, RIKEN full-length enriched library, |
| | clone: 4933433M02 product: RAD54 like (S. cerevisiae), full insert sequence. |
| BG071837 | H3103G03-3 NIA Mouse 15K cDNA Clone Set Mus musculus cDNA clone |
| | H3103G03 3′, mRNA sequence. |
| BB320205 | Transcribed sequences |
| BB357976 | RIKEN cDNA 4930438M06 gene |
| BG066611 | Transcribed sequences |
| AA175473 | LOC381118 (LOC381118), mRNA |
| BB373408 | kringle containing transmembrane protein |
| BM201500 | Transcribed sequences |
| BB014626 | expressed sequence AI597013 |
| BF152799 | RIKEN cDNA 1700121K02 gene |
| BC026654 | cDNA sequence BC018222 |
| AW050002 | RIKEN cDNA 1110035L05 gene |
| D78270 | golgi autoantigen, golgin subfamily a, 3 |
| BI646094 | 603274893F1 NCI_CGAP_Mam3 Mus musculus cDNA clone IMAGE: 5315422 5′, |
| | mRNA sequence. |
| BB128085 | Transcribed sequences |
| AV222756 | AV222756 RIKEN full-length enriched, 18 days pregnant, placenta and extra |
| | embryonic tissue Mus musculus cDNA clone 3830404B07 3′, mRNA sequence. |
| BB487918 | thiamin pyrophosphokinase |
| AK017976 | Adult male thymus cDNA, RIKEN full-length enriched library, clone: 5830437K03 |
| | product: unclassifiable, full insert sequence |
| BC024577 | RIKEN cDNA 4933426I21 gene |
| BQ175967 | Similar to hypothetical protein FLJ36766 (LOC277743), mRNA |
| AI649303 | uk30d11.x1 Sugano mouse kidney mkia Mus musculus cDNA clone IMAGE: 1970517 |
| | 3′, mRNA sequence. |
| BB164652 | BB164652 RIKEN full-length enriched, 16 days neonate thymus Mus musculus |
| | cDNA clone A130079E13 3′, mRNA sequence. |
| AA162958 | RIKEN cDNA 2200001I15 gene |
| BE446953 | Transcribed sequence with moderate similarity to protein sp: O14775 (H. sapiens) |
| | GBB5_HUMAN Guanine nucleotide-binding protein beta subunit 5 |
| BB488001 | DNA segment, Chr 3, ERATO Doi 330, expressed |
| BM121861 | vacuolar protein sorting 54 (yeast) |
| BB368030 | expressed sequence AI413414 |
| AV278800 | AV278800 RIKEN full-length enriched, adult male testis (DH10B) Mus musculus |
| | cDNA clone 4933403O03 3′, mRNA sequence. |
| BB332426 | myristoylated alanine rich protein kinase C substrate |
| NM_008987 | pentaxin related gene |
| BB551855 | Transcribed sequences |
| NM_008834 | expressed sequence AA939927 |
| AA940525 | vz45f09.r1 Soares_thymus_2NbMT Mus musculus cDNA clone IMAGE: 1329449 5′ |
| | similar to SW: NIGM_BOVIN Q02374 NADH-UBIQUINONE OXIDOREDUCTASE |
| | AGGG SUBUNIT PRECURSOR;, mRNA sequence. |
| BB441985 | CDC14 cell division cycle 14 homolog A (S. cerevisiae) |
| AI849219 | Transcribed sequences |
| AW556597 | Adult male testis cDNA, RIKEN full-length enriched library, clone: 1700023H06 |
| | product: unknown EST, full insert sequence |
| BM218981 | pleckstrin homology domain interacting protein |
| BB223866 | Adult male aorta and vein cDNA, RIKEN full-length enriched library, |
| | clone: A530084B03 product: unknown EST, full insert sequence |
| BM223619 | Transcribed sequences |
| NM_033314 | solute carrier organic anion transporter family, member 2a1 |
| BE372017 | 601223607F1 NCI_CGAP_Mam1 Mus musculus cDNA clone IMAGE: 3582145 5′, |
| | mRNA sequence. |
| AK016730 | RIKEN cDNA 4933407L21 gene |
| AK005680 | DnaJ (Hsp40) homolog, subfamily B, member 6 |
| AU043156 | Transcribed sequences |
| AV038578 | Transcribed sequence with strong similarity to protein pir: T51146 (H. sapiens) |
| | T51146 ring-box protein 1 [imported] - human |
| AK015425 | RIKEN cDNA 4930538D17 gene |
| BC005410 | nuclear respiratory factor 1 |
| BC014723 | phosphodiesterase 6G, cGMP-specific, rod, gamma |
| BB181330 | Adult male hypothalamus cDNA, RIKEN full-length enriched library, |
| | clone: A230090L04 product: unknown EST, full insert sequence |
| BC027185 | RIKEN cDNA 2210023G05 gene |
| BB177090 | RIKEN cDNA 2610511E03 gene |
| AV339322 | AV339322 RIKEN full-length enriched, adult male olfactory bulb Mus musculus |
| | cDNA clone 6430503O04 3′, mRNA sequence. |
| BB200000 | 0 day neonate thymus cDNA, RIKEN full-length enriched library, clone: A430019C16 |
| | product: unknown EST, full insert sequence |
| BB429200 | vacuolar protein sorting 52 (yeast) |
| BE980134 | RIKEN cDNA 4930534B04 gene |
| BC018365 | immunoglobulin heavy chain 1a (serum IgG2a) |
| BB519382 | BB519382 RIKEN full-length enriched, 16 days neonate heart Mus musculus cDNA |
| | clone D830035N10 3′ similar to AB023966 Rattus norvegicus mRNA for Rod1, |
| | mRNA sequence. |
| AK015789 | cellular nucleic acid binding protein 2 |
| BB295128 | RIKEN cDNA A230098A12 gene |
| BB138441 | RIKEN cDNA A730089K16 gene |
| NM_027622 | RIKEN cDNA 4921530G04 gene |
| BQ034009 | DEAD (Asp-Glu-Ala-Asp) box polypeptide 6 |
| BB782615 | BB782615 RIKEN full-length enriched, Nullipotent stem cell CRL-2070 NE cDNA |
| | Mus musculus cDNA clone G430082E04 3′, mRNA sequence. |
| BB292344 | BB292344 RIKEN full-length enriched, 9.5 days embryo parthenogenote Mus |
| | musculus cDNA clone B130010I10 3′, mRNA sequence. |
| BB427897 | ankyrin 2, brain |
| AV001099 | EST C77032 |
| BB558642 | BB558642 RIKEN full-length enriched, 2 days pregnant adult female ovary Mus |
| | musculus cDNA clone E330035D17 3′, mRNA sequence. |
| BB462562 | Musashi homolog 1(Drosophila) |
| BB324138 | LISCH7-like |
| BB754492 | cell division cycle 27 homolog (S. cerevisiae) |
| AB050203 | calpain 8 |
| NM_013875 | phosphodiesterase 7B |
| NM_009154 | sema domain, seven thrombospondin repeats (type 1 and type 1-like), |
| | transmembrane domain (TM) and short cytoplasmic domain, (semaphorin) 5A |
| AF426024 | serine (or cysteine) proteinase inhibitor, clade B, member 1a |
| NM_013626 | peptidylglycine alpha-amidating monooxygenase |
| BF460623 | RAN GTPase activating protein 1 |
| AK011603 | breast carcinoma amplified sequence 3 |
| BB103682 | 12 days embryo embryonic body between diaphragm region and neck cDNA, RIKEN |
| | full-length enriched library, clone: 9430087N24 product: unknown EST, full insert |
| | sequence |
| BB332449 | BB332449 RIKEN full-length enriched, 6 days neonate medulla oblongata Mus |
| | musculus cDNA clone B730048G11 3′, mRNA sequence. |
| BM120811 | Transcribed sequence with moderate similarity to protein pir.S12207 (M. musculus) |
| | S12207 hypothetical protein |
| BF018114 | crystallin, alpha B |
| U15647 | |
| BF318506 | RIKEN cDNA 1700023B23 gene |
| BB476065 | hypothetical protein LOC217831 |
| BE285361 | 601091410F1 NCI_CGAP_Mam5 Mus musculus cDNA clone IMAGE: 3485906 5′, |
| | mRNA sequence. |
| BB775592 | RIKEN cDNA 1200020A08 gene |
| BF018351 | RIKEN cDNA 1110013G13 gene |
| BE980451 | Transcribed sequences |
| BC024674 | RIKEN cDNA 4633402D15 gene |
| NM_008166 | glutamate receptor, ionotropic, delta 1 |
| BC023100 | RIKEN cDNA 1810044O22 gene |
| BB533744 | Transcribed sequences |
| BB084614 | RIKEN cDNA 2310046G15 gene |
| BM238926 | 9 days embryo whole body cDNA, RIKEN full-length enriched library, |
| | clone: D030050E20 product: unclassifiable, full insert sequence |
| NM_007652 | CD59a antigen |
| BC025127 | Rho guanine nucleotide exchange factor (GEF) 5 |
| AB031037 | eomesodermin homolog (Xenopus laevis) |
| BB772341 | Transcribed sequence with strong similarity to protein sp: P00722 (E. coli) |
| | BGAL_ECOLI Beta-galactosidase |
| AW550676 | Transcribed sequences |
| AK020456 | RIKEN cDNA 9430034F23 gene |
| AK016992 | Adult male testis cDNA, RIKEN full-length enriched library, clone: 4933430H06 |
| | product: unclassifiable, full insert sequence |
| NM_009380 | thyroid hormone receptor beta |
| NM_013596 | melanocortin 5 receptor |
| AF127140 | fibroblast growth factor receptor 4 |
| BB775176 | BB775176 RIKEN full-length enriched, brain CRL-1443 BC3H1 cDNA Mus |
| | musculus cDNA clone G430023N11 3′, mRNA sequence. |
| NM_026533 | ribosomal protein S13 |
| C77296 | Transcribed sequence with weak similarity to protein ref: NP_035070.1 |
| | (M. musculus) non-selective cation channel 1 [Mus musculus] |
| C80147 | hepatoma-derived growth factor |
| AV015526 | RIKEN cDNA 1110032E16 gene |
| BM213778 | Transcribed sequence with moderate similarity to protein ref: NR_060291.1 |
| | (H. sapiens) hypothetical protein FLJ20435 [Homo sapiens] |
| AW123906 | 3 days neonate thymus cDNA, RIKEN full-length enriched library, |
| | clone: A630023D23 product: hypothetical Ankyrin repeat region circular profile |
| | containing protein, full insert sequence |
| AK015672 | CUB and Sushi multiple domains 3 |
| BB308198 | RIKEN cDNA A930014I12 gene |
| BE956260 | UI-M-BH4-baw-h-10-0-UI.s1 NIH_BMAP_M_S5 Mus musculus cDNA clone UI-M- |
| | BH4-baw-h-10-0-UI 3′, mRNA sequence. |
| BE627942 | 16 days embryo head cDNA, RIKEN full-length enriched library, clone: C130094K23 |
| | product: hypothetical protein, full insert sequence |
| BB528056 | RIKEN cDNA C630015B17 gene |
| BE307471 | RIKEN cDNA 1110055N21 gene |
| BB251739 | BB251739 RIKEN full-length enriched, 7 days neonate cerebellum Mus musculus |
| | cDNA clone A730047M15 3′ similar to D29766 Rattus norvegicus mRNA for Crk- |
| | associated substrate, p130, mRNA sequence. |
| AV258074 | RIKEN cDNA 1700013E18 gene |
| AK002930 | RIKEN cDNA 2610104C07 gene |
| AV045829 | AV045829 Mus musculus adult C57BL/6J testis Mus musculus cDNA clone |
| | 1700049A13, mRNA sequence. |
| AK015573 | RIKEN cDNA 4930474F22 gene |
| BE456545 | RIKEN cDNA 2810442I21 gene |
| BB733992 | Transcribed sequences |
| BB043509 | Transcribed sequences |
| BB252309 | 7 days neonate cerebellum cDNA, RIKEN full-length enriched library, |
| | clone: A730050L22 product: unknown EST, full insert sequence |
| AK018072 | 14 days embryo thymus cDNA, RIKEN full-length enriched library, |
| | clone: 6130400H19 product: unknown EST, full insert sequence |
| AK009188 | RIKEN cDNA 2310006M14 gene |
| BB450104 | troponin T1, skeletal, slow |
| AW536432 | heparan sulfate 6-O-sulfotransferase 2 |
| NM_011202 | protein tyrosine phosphatase, non-receptor type 11 |
| BG067824 | Transcribed sequences |
| AW061092 | Similar to solute carrier family 9 (sodium/hydrogen exchanger), isoform 5 |
| | (LOC277973), mRNA |
| BB140360 | Transcribed sequences |
| BG070088 | Transcribed sequences |
| BB364080 | 16 days embryo head cDNA, RIKEN full-length enriched library, clone: C130020A17 |
| | product: unknown EST, full insert sequence |
| AV377077 | AV377077 RIKEN full-length enriched, adult male cecum Mus musculus cDNA |
| | clone 9130221L21 3′, mRNA sequence. |
| NM_008499 | LIM homeobox protein 5 |
| BB105328 | high mobility group AT-hook 2 |
| U73626 | potassium inwardly rectifying channel, subfamily J, member 11 |
| AK011230 | RIKEN cDNA 2600016L03 gene |
| BG0B1571 | H3066F10-5 NIA Mouse 15K cDNA Clone Set Mus musculus cDNA clone |
| | H3066F10 5′, mRNA sequence. |
| AK017490 | M-phase phosphoprotein 10 (U3 small nucleolar ribonucleoprotein) |
| AF349142 | immunoglobulin heavy chain 4 (serum IgG1) |
| AI843088 | Adult male spinal cord cDNA, RIKEN full-length enriched library, clone: A330096I21 |
| | product: weakly similar to RETBINDIN [Homo sapiens], full insert sequence |
| NM_010168 | coagulation factor II |
| AA396586 | membrane-associated protein 17 |
| NM_009152 | sema domain, immunoglobulin domain (Ig), short basic domain, secreted, |
| | (semaphorin) 3A |
| BB042885 | RIKEN cDNA 9830141C09 gene |
| AK006136 | RIKEN cDNA 1700019O17 gene |
| AW123011 | 13 days embryo liver cDNA, RIKEN full-length enriched library, clone: 2510003B16 |
| | product: unknown EST, full insert sequence |
| NM_010068 | DNA methyltransferase 3B |
| BG065987 | H3037F05-3 NIA Mouse 15K cDNA Clone Set Mus musculus cDNA clone |
| | H3037F05 3′, mRNA sequence. |
| BB283533 | dedicator of cyto-kinesis 1 |
| BC010248 | RIKEN cDNA 1810048P08 gene |
| BB560335 | Transcribed sequences |
| NM_009202 | solute carrier family 22 (organic cation transporter), member 1 |
| NM_013671 | superoxide dismutase 2, mitochondrial |
| BM934562 | syntaxin 5A |
| BB208212 | phosphatidylinositol 4-kinase type 2 alpha |
| AV206400 | Adult male testis cDNA, RIKEN full-length enriched library, clone: 1700007J24 |
| | product: unknown EST, full insert sequence |
| AK015570 | Mus musculus adult male testis cDNA, RIKEN full-length enriched library, |
| | clone: 4930474A20 product: unclassifiable, full insert sequence. |
| AV227581 | claudin 1 |
| BB524597 | Kruppel-like factor 7 (ubiquitous) |
| AK014924 | Adult male testis cDNA, RIKEN full-length enriched library, clone: 4921519G19 |
| | product: unclassifiable, full insert sequence |
| AK009368 | RIKEN cDNA 2310015L07 gene |
| AI505185 | RIKEN cDNA 2410091N08 gene |
| BG066733 | H3046C11-3 NIA Mouse 15K cDNA Clone Set Mus musculus cDNA clone |
| | H3046C11 3′, mRNA sequence. |
| AK017594 | leucine rich repeat and fibronectin type III domain containing 2 |
| BB499286 | expressed sequence AI427100 |
| AI503297 | Transcribed sequence with weak similarity to protein ref: NP_081764.1 |
| | (M. musculus) RIKEN cDNA 5730493B19 [Mus musculus] |
| BI076724 | Transcribed sequence with moderate similarity to protein pir: S12207 (M. musculus) |
| | S12207 hypothetical protein |
| BB224305 | cyclin E2 |
| BB391868 | expressed sequence AW123240 |
| NM_011661 | tyrosinase |
| BB400711 | ES cells cDNA, RIKEN full-length enriched library, clone: C330019L16 |
| | product: weakly similar to SIMILAR TO ZINC FINGER PROTEIN 14 (KOX 6) [Mus |
| | musculus], full insert sequence |
| AF093257 | homer homolog 1 (Drosophila) |
| BE951353 | RIKEN cDNA 1500005I02 gene |
| BG092264 | Transcribed sequence with weak similarity to protein ref: NP_081764.1 |
| | (M. musculus) RIKEN cDNA 5730493B19 [Mus musculus] |
| BB469133 | RIKEN cDNA 1810057P16 gene |
| BB244040 | Transcribed sequences |
| AK019240 | SCY1-like 1 (S. cerevisiae) |
| AI627121 | Transcribed sequences |
| NM_010473 | histidine rich calcium binding protein |
| AF156549 | ATPase, class V, type 10A |
| NM_028402 | |
| NM_010512 | insulin-like growth factor 1 |
| AK009208 | 10 days neonate cerebellum cDNA, RIKEN full-length enriched library, |
| | clone: B930089N03 product: hypothetical Gag gene protein p24 (core nucleocapsid |
| | protein) containing protein, full insert sequence |
| BB306699 | Adult male corpora quadrigemina cDNA, RIKEN full-length enriched library, |
| | clone: B230209D03 product: unknown EST, full insert sequence |
| BG069765 | RIKEN cDNA 5830435C13 gene |
| BG071961 | H3105B06-3 NIA Mouse 15K cDNA Clone Set Mus musculus cDNA clone |
| | H3105B06 3′, mRNA sequence. |
| AK017407 | carbohydrate (N-acetylgalactosamine 4-0) sulfotransferase 9 |
| NM_133353 | oocyte secreted protein 1 |
| BC021367 | cDNA sequence BC021367 |
| BM939297 | Mpv17 transgene, kidney disease mutant |
| BB590675 | paraspeckle protein 1 |
| BG075885 | Hypothetical LOC327992 (LOC327992), mRNA |
| BC021484 | sarcospan |
| AV254985 | RIKEN cDNA 4921515A04 gene |
| C76618 | sterol carrier protein 2, liver |
| AW557616 | Adult male aorta and vein cDNA, RIKEN full-length enriched library, |
| | clone: A530084D13 product: unknown EST, full insert sequence |
| BM213143 | RIKEN cDNA 2700083B06 gene |
| AA408781 | DNA segment, Chr 5, Brigham & Women's Genetics 1524 expressed |
| BB376471 | wee 1 homolog (S. pombe) |
| BB132726 | 15 days embryo head cDNA, RIKEN full-length enriched library, clone: D930008A07 |
| | product: unknown EST, full insert sequence |
| NM_028227 | BRCA1 associated protein |
| NM_007523 | BCL2-antagonist/killer 1 |
| AI327364 | zeta-chain (TCR) associated protein kinase |
| BE133651 | stearoyl-coenzyme A desaturase 3 |
| AK005880 | RIKEN cDNA 1700029H01 gene |
| BM240058 | expreexpressed sequence AA408868 |
| AK010021 | abhydrolase domain containing 9 |
| BM119782 | RIKEN cDNA D630044D05 gene |
| BC018414 | heat shock factor 2 |
| BB443609 | cDNA sequence BC027828 |
| AK018062 | 12 days embryo spinal ganglion cDNA, RIKEN full-length enriched library, |
| | clone: D130074J01 product: unclassifiable, full insert sequence |
| BG066667 | Transcribed sequences |
| NM_009955 | dihydropyrimidinase-like 2 |
| AI666576 | mu15d07.x1 Soares_thymus_2NbMT Mus musculus cDNA clone IMAGE: 639469 3′, |
| | mRNA sequence. |
| AF354666 | cytochrome b reductase 1 |
| NM_023884 | RIKEN cDNA 4921528G01 gene |
| BB424644 | RIKEN cDNA 6030468D11 gene |
| AK016341 | RIKEN cDNA 5133400G04 gene |
| AV268106 | RIKEN cDNA 4930405J24 gene |
| BM195384 | 13 days embryo heart cDNA, RIKEN full-length enriched library, clone: D330024D06 |
| | product: unknown EST, full insert sequence |
| BB392041 | BB392041 RIKEN full-length enriched, 0 day neonate cerebellum Mus musculus |
| | cDNA clone C230077J16 3′, mRNA sequence. |
| BB030565 | zinc finger protein 282 |
| BG803764 | 10 days neonate cerebellum cDNA, RIKEN full-length enriched library, |
| | clone: B930092N05 product: unknown EST, full insert sequence |
| AK006467 | RIKEN cDNA 1700028N11 gene |
| BE624323 | DnaJ (Hsp40) homolog, subfamily C, member 3 |
| NM_018865 | WNT1 inducible signaling pathway protein 1 |
| NM_007390 | cholinergic receptor, nicotinic, alpha polypeptide 7 |
| BC020991 | lipase, endothelial |
| BG070330 | transmembrane channel-like gene family 7 |
| BB349535 | RIKEN cDNA 1810044A24 gene |
| BB229373 | Transcribed sequences |
| NM_010290 | gap junction membrane channel protein alpha 9 |
| BG072298 | Transcribed sequence with weak similarity to protein ref: NP_081764.1 |
| | (M. musculus) RIKEN cDNA 5730493B19 [Mus musculus] |
| NM_007413 | adenosine A2b receptor |
| BB200603 | DNA-damage inducible transcript 3 |
| BE949045 | Transcribed sequences |
| AW551808 | RIKEN cDNA 6820443O06 gene |
| AF419324 | calcium binding protein 7 |
| AF479673 | purine-rich element binding protein G |
| BB427699 | RIKEN cDNA 2410077I05 gene |
| BB667439 | BRAF35/HDAC2 complex |
| AV375182 | solute carrier organic anion transporter family, member 4a1 |
| AV310220 | RAD54 like (S. cerevisiae) |
| BB378019 | BB378019 RIKEN full-length enriched, 16 days embryo head Mus musculus cDNA |
| | clone C130092C09 3′, mRNA sequence. |
| BB333039 | Transcribed sequences |
| BB337425 | RIKEN cDNA 4931426K16 gene |
| BB815530 | kit ligand |
| AU043080 | Transcribed sequences |
| AK017758 | RIKEN cDNA 5730508B09 gene |
| W45978 | Clone IMAGE: 4206343, mRNA |
| BB019018 | Transcribed sequence with moderate similarity to protein ref: NP_067735.1 |
| | (R. norvegicus) Testis-specific A-kinase-anchoring-protein [Rattus norvegicus] |
| BB487289 | ATPase, class V, type 10A |
| AK012005 | RIKEN cDNA 5430405H02 gene |
| AI324734 | Similar to calreticulin 3 (LOC381124), mRNA |
| BB172347 | Adult male hypothalamus cDNA, RIKEN full-length enriched library, |
| | clone: A230038B01 product: unclassifiable, full insert sequence |
| AK016514 | meiosis defective 1 |
| BB313728 | Adult male diencephalon cDNA, RIKEN full-length enriched library, |
| | clone: 9330127N12 product: PROTEIN KINASE C-BINDING PROTEIN NELL1 |
| | PRECURSOR (NEL-LIKE PROTEIN 1) homolog [Rattus norvegicus], full insert |
| | sequence |
| NM_007439 | anaplastic lymphoma kinase |
| AV252141 | RIKEN cDNA 2410005O16 gene |
| NM_134155 | breast cancer metastasis-suppressor 1 |
| C77540 | Transcribed sequences |
| AI844428 | 16 days neonate cerebellum cDNA, RIKEN full-length enriched library, |
| | clone: 9630010L05 product: unclassifiable, full insert sequence |
| AV360460 | AV360460 RIKEN full-length enriched, adult male eyeball Mus musculus cDNA |
| | clone 7530412M05 3′, mRNA sequence. |
| BB027977 | Transcribed sequences |
| C79741 | Transcribed sequence with weak similarity to protein prf: 1614337A (M. musculus) |
| | 1614337A formin [Mus musculus] |
| C77581 | C77581 Mouse 3.5-dpc blastocyst cDNA Mus musculus cDNA clone J0034A10 3′, |
| | mRNA sequence. |
| AI385532 | thrombospondin 1 |
| BB181350 | Adult male urinary bladder cDNA, RIKEN full-length enriched library, |
| | clone: 9530064D18 product: unknown EST, full insert sequence |
| BB375206 | 16 days embryo head cDNA, RIKEN full-length enriched library, clone: C130078N14 |
| | product: unknown EST, full insert sequence |
| BB280444 | Adult retina cDNA, RIKEN full-length enriched library, clone: A930026H04 |
| | product: hypothetical protein, full insert sequence |
| BB180990 | Adult male hypothalamus cDNA, RIKEN full-length enriched library, |
| | clone: A230088E03 product: unknown EST, full insert sequence |
| BG067340 | cholinergic receptor, nicotinic, alpha polypeptide 5 |
| BB127813 | RIKEN cDNA A830010M20 gene |
| NM_007823 | cytochrome P450, family 4, subfamily b, polypeptide 1 |
| AV328340 | AV328340 RIKEN full-length enriched, adult male medulla oblongata Mus musculus |
| | cDNA clone 6330441E06 3′, mRNA sequence. |
| BB758406 | CDNA clone IMAGE: 3825663, partial cds |
| BB560802 | BB560802 RIKEN full-length enriched, 10 days neonate olfactory brain Mus |
| | musculus cDNA clone E530116F06 3′, mRNA sequence. |
| X94151 | chemokine (C—C motif) receptor 5 |
| AK020544 | RIKEN cDNA 9530004P13 gene |
| BB314393 | Transcribed sequence |
| BG067256 | ubiquitin specific protease 16 |
| D19363 | implantation serine protease 2 |
| AK009650 | RIKEN cDNA 2310036I02 gene |
| NM_030690 | retinoic acid induced 14 |
| BB369687 | RIKEN cDNA A130001D14 gene |
| AK018666 | cysteine-rich motor neuron 1 |
| AK006130 | AXIN1 up-regulated 1 |
| BG083186 | RIKEN cDNA 0610027O18 gene |
| AK011843 | Mus musculus 10 days embryo whole body cDNA, RIKEN full-length enriched |
| | library, clone: 2610111C21 product: TAF15 RNA polymerase II, TATA box binding |
| | protein (TBP)-associated factor, 68 kDa, full insert sequence. |
| BB044088 | 13 days embryo male testis cDNA, RIKEN full-length enriched library, |
| | clone: 6030481K23 product: unclassifiable, full insert sequence |
| AK017188 | 11 days pregnant adult female ovary and uterus cDNA, RIKEN full-length enriched |
| | library, clone: 5033423K11 product: hypothetical protein, full insert sequence |
| NM_007984 | fascin homolog 1, actin bundling protein (Strongylocentrotus) purpuratus) |
| BM570681 | 18-day embryo whole body cDNA, RIKEN full-length enriched library, |
| | clone: 1110059G02 product: unclassifiable, full insert sequence |
| BB053109 | BB053109 RIKEN full-length enriched, 12 days embryo male wolffian duct Mus |
| | musculus cDNA clone 6720460B08 3′, mRNA sequence. |
| AK009247 | WD repeat domain 5B |
| BB384542 | tripartite motif protein 33 |
| AW537496 | histone cell cycle regulation defective homolog A (S. cerevisiae) |

TABLE 2

| Genbank No. | Gene Title |
|---|---|
| AF022856 | neuropilin 2 |
| NM_015825 | SH3-binding domain glutamic acid-rich protein |
| BB042892 | RIKEN cDNA 6030424L22 gene |
| U96702 | similar to human proteinase inhibitor 8; Mus musculus serine proteinase inhibitor |
| | mBM17 mRNA, partial cds. |
| AV319357 | a disintegrin-like and metalloprotease (reprolysin type) with thrombospondin type 1 |
| | motif, 16 |
| BB039073 | Transcribed sequence with moderate similarity to protein ref: NP_036135.1 |
| | (M. musculus) cofactor required for Sp1 transcriptional activation subunit 2 |
| BE692283 | Adult male corpus striatum cDNA, RIKEN full-length enriched library, |
| | clone: C030013G03 product: unknown EST, full insert sequence |
| BB075807 | RIKEN cDNA 4732418C07 gene |
| AV375098 | RIKEN cDNA 9130020L07 gene |
| BF319769 | bobby sox homolog (Drosophila) |
| BE948730 | prostaglandin E receptor 1 (subtype EP1) |
| BB145092 | Transcribed sequences |
| X57960 | ribosomal protein L7 |
| AW538733 | Transcribed sequence with moderate similarity to protein pir: S12207 (M. musculus) |
| | S12207 hypothetical protein |
| M25487 | histone 1, H2bp |
| AV373944 | cytochrome b-245, beta polypeptide |
| BE623057 | Transcribed sequences |
| AK002682 | RIKEN cDNA 0610027B03 gene |
| AK008801 | unnamed protein product; CGI-94 PROTEIN homolog [Homo sapiens] |
| | (SPTR\|Q9BS98, evidence: FASTY, 90.5% ID, 100% length, match = 759) putative; Mus |
| | musculus adult male stomach cDNA, RIKEN full-length enriched library, |
| | clone: 2210402H06 product: CGI-94 PROTEIN homolog [Homo sapiens], full insert |
| | sequence. |
| BB105776 | RIKEN cDNA 4921525L17 gene |
| AI747732 | Transcribed sequences |
| AK008753 | RIKEN cDNA 2210019E14 gene |
| NM_011402 | solute carrier family 34 (sodium phosphate), member 2 |
| BG065782 | cysteine-rich hydrophobic domain 1 |
| AV010327 | SET and MYND domain containing 1 |
| BB460605 | ceroid-lipofuscinosis, neuronal 8 |
| BG066927 | H3048F03-3 NIA Mouse 15K cDNA Clone Set Mus musculus cDNA clone H3048F03 |
| | 3′, mRNA sequence. |
| BB325197 | Transcribed sequences |
| BB400773 | RIKEN cDNA 5630401H01 gene |
| BB042052 | 13 days embryo male testis cDNA, RIKEN full-length enriched library, |
| | clone: 6030459M14 product: unclassifiable, full insert sequence |
| BB522283 | expressed sequence AA986860 |
| AV274006 | hypothetical protein 4932415O19 |
| NM_010940 | non-selective cation channel 1 |
| BB745929 | RIKEN cDNA D430015B01 gene |
| AV152299 | Adult male olfactory brain cDNA, RIKEN full-length enriched library, |
| | clone: 6430510H04 product: unclassifiable, full insert sequence |
| NM_009369 | transforming growth factor, beta induced |
| AV369536 | six transmembrane epithelial antigen of prostate 2 |
| AK021022 | Mus musculus 4 days neonate male adipose cDNA, RIKEN full-length enriched |
| | library, clone: B430309A01 product: exportin 4, full insert sequence. |
| NM_023132 | renin binding protein |
| AV297945 | myosin X |
| NM_016904 | CDC28 protein kinase 1 |
| AI846902 | Transcribed sequences |
| BB496614 | BB496614 RIKEN full-length enriched, 0 day neonate kidney Mus musculus cDNA |
| | clone D630003P16 3′, mRNA sequence. |
| BB366293 | 16 days embryo head cDNA, RIKEN full-length enriched library, clone: C130033B14 |
| | product: unknown EST, full insert sequence |
| U95921 | Adult male thymus cDNA, RIKEN full-length enriched library, clone: 5830404G23 |
| | product: T-cell receptor alpha chain precursor V-J region (TA72) (fragment) homolog |
| | [Mus musculus], full insert sequence |
| AA517021 | Transcribed sequences |
| AK005893 | allantoicase |
| BB327301 | expressed sequence AI604832 |
| BB536333 | RIKEN cDNA E030049G20 gene |
| BB056038 | BB056038 RIKEN full-length enriched, 12 days embryo male wolffian duct Mus |
| | musculus cDNA clone 6720487A17 3′, mRNA sequence. |
| BM122872 | nuclear factor of activated T-cells, cytoplasmic, calcineurin-dependent 2 |
| BB667693 | DNA segment, Chr 7, Brigham & Women's Genetics 0421 expressed |
| AI462171 | Transcribed sequence with weak similarity to protein pir: RGECDW (E. coli) RGECDW |
| | transcription activator of D-serine dehydratase - Escherichia coli |
| BC024402 | RIKEN cDNA A430103C15 gene |
| AW125804 | myocilin |
| NM_053147 | protocadherin beta 22 |
| AK017673 | RIKEN cDNA 2810417H13 gene |
| BB757349 | zinc finger CCCH type, antiviral 1 |
| BB053540 | 12 days embryo male wolffian duct includes surrounding region cDNA, RIKEN full- |
| | length enriched library, clone: 6720464D04 product: unknown EST, full insert sequence |
| BB280137 | RAB guanine nucleotide exchange factor (GEF) 1 |
| AI854375 | UI-M-BG1-aic-f-07-0-UI.s1 NIH_BMAP_MSC_N Mus musculus cDNA clone UI-M- |
| | BG1-aic-f-07-0-UI 3′, mRNA sequence. |
| AF334736 | glutamine fructose-6-phosphate transaminase 1 |
| BB325766 | RIKEN cDNA B430119L13 gene |
| AK018117 | RIKEN cDNA 0610016O18 gene |
| BB204648 | Transcribed sequences |
| BC025840 | titin |
| BM194997 | RIKEN cDNA 6030443O07 gene |
| BG063310 | H3005F05-3 NIA Mouse 15K cDNA Clone Set Mus musculus cDNA clone H3005F05 |
| | 3′, mRNA sequence. |
| BB548441 | RIKEN cDNA 4930538K18 gene |
| NM_010008 | cytochrome P450, family 2, subfamily j, polypeptide 6 |
| BB434779 | microtubule-associated protein, RP/EB family, member 2 |
| AK010351 | cell division cycle associated 1 |
| BG073838 | Transcribed sequences |
| BM115172 | p300/CBP-associated factor |
| BI684288 | phosphoinositide-3-kinase adaptor protein 1 |
| BB375943 | dual specificity phosphatase 12 |
| BC021340 | cDNA sequence BC021340 |
| BB519728 | RIKEN cDNA 2900006B13 gene |
| NM_010232 | flavin containing monooxygenase 5 |
| NM_052823 | FXYD domain-containing ion transport regulator 2 |
| BM124141 | SERTA domain containing 3 |
| BB044002 | RIKEN cDNA 6330406P08 gene |
| BF454057 | potassium channel tetramerisation domain containing 12b |
| AK011162 | RIKEN cDNA 2600005O03 gene |
| BM202770 | cysteine rich protein 61 |
| BB250200 | RIKEN cDNA 1110059P08 gene |
| BB209663 | Transcribed sequences |
| BB746075 | dipeptidylpeptidase 7 |
| BB445665 | RIKEN cDNA D030051N19 gene |
| BG143672 | Transcribed sequences |
| NM_133978 | chemokine-like factor super family 7 |
| BB635017 | 0 day neonate head cDNA, RIKEN full-length enriched library, clone: 4833440O21 |
| | product: unknown EST, full insert sequence |
| BB528391 | NIMA (never in mitosis gene a)-related expressed kinase 6 |
| AF065917 | leukemia inhibitory factor |
| BB076798 | BB076798 RIKEN full-length enriched, adult male diencephalon Mus musculus cDNA |
| | clone 9330128J04 3′, mRNA sequence. |
| NM_020612 | cell adhesion regulator; go_component: cellular_component unknown [goid 0008372] |
| | [evidence ND]; go_function: cell adhesion receptor regulator activity [goid 0042064] |
| | [evidence TAS] [pmid 8611648]; go_process: cell adhesion [goid 0007155] [evidence |
| | TAS] [pmid 8611648]; Mus musculus cell matrix adhesion regulator (Cmar), mRNA. |
| AK021006 | stress 70 protein chaperone, microsome-associated, human homolog |
| BB822050 | BB822050 RIKEN full-length enriched, mammary gland RCB-0526 Jyg-MC(A) cDNA |
| | Mus musculus cDNA clone G830016N13 3′, mRNA sequence. |
| BB773386 | Hedgehog-interacting protein |
| NM_011075 | ATP-binding cassette, sub-family B (MDR/TAP), member 1B |
| BM228837 | RIKEN cDNA 6030401B09 gene |
| BB198496 | nudix (nucleoside diphosphate linked moiety X)-type motif 15 |
| BI133940 | Transcribed sequences |
| BB652005 | DNA segment, Chr 8, ERATO Doi 69, expressed |
| BB220625 | Adult male aorta and vein cDNA, RIKEN full-length enriched library, |
| | clone: A530061A19 product: unknown EST, full insert sequence |
| AV101011 | AV101011 Mus musculus C57BL/6J ES cell Mus musculus cDNA clone 2410070L23, |
| | mRNA sequence. |
| BB181247 | myelin basic protein |
| BB231078 | cytidine and dCMP deaminase domain containing 1 |
| AV313371 | RIKEN cDNA C030046M14 gene |
| BB653800 | Adult male liver tumor cDNA, RIKEN full-length enriched library, clone: C730026I01 |
| | product: unknown EST, full insert sequence |
| BB500282 | Transcribed sequences |
| AV332635 | AV332635 RIKEN full-length enriched, adult male medulla oblongata Mus musculus |
| | cDNA clone 6330536E12 3′, mRNA sequence. |
| BB797251 | Transcribed sequences |
| BB485233 | Transcribed sequences |
| U58494 | |
| AK019901 | Adult male pituitary gland cDNA, RIKEN full-length enriched library, |
| | clone: 5330421F07 product: hypothetical Calponin homology (CH) domain containing |
| | protein, full insert sequence |
| BB797985 | Transcribed sequences |
| BM244106 | Spi-B transcription factor (Spi-1/PU.1 related) |
| AK010086 | nuclear factor I/B |
| AK004227 | RIKEN cDNA 1110051B16 gene |
| BB241535 | suppressor of cytokine signaling 3 |
| BB636024 | Transcribed sequences |
| BB209878 | BB209878 RIKEN full-length enriched, 0 day neonate thymus Mus musculus cDNA |
| | clone A430094K05 3′, mRNA sequence. |
| AV300228 | expressed sequence AW491445 |
| AU018569 | RIKEN cDNA C330027C09 gene |
| AV297961 | RIKEN cDNA 5730453H04 gene |
| BB319478 | BB319478 RIKEN full-length enriched, adult male corpora quadrigemina Mus |
| | musculus cDNA clone B230380I21 3′, mRNA sequence. |
| BB283832 | Transcribed sequences |
| BB453864 | Transcribed sequences |
| BC003738 | RAD51 associated protein 1 |
| BB396783 | 0 day neonate cerebellum cDNA, RIKEN full-length enriched library, |
| | clone: C230099C02 product: unknown EST, full insert sequence |
| AI605450 | vo17d02.x1 Barstead mouse myotubes MPLRB5 Mus musculus cDNA clone |
| | IMAGE: 1050147 3′, mRNA sequence. |
| BC008159 | RIKEN cDNA 2810432O22 gene |
| BI104953 | Adult male aorta and vein cDNA, RIKEN full-length enriched library, |
| | clone: A530060J09 product: hypothetical protein, full insert sequence |
| BE283373 | 601103319F1 NCI_CGAP_Lu29 Mus musculus cDNA clone IMAGE: 3495557 5′, mRNA |
| | sequence. |
| BC008104 | claudin 7 |
| AI481778 | vesicle-associated membrane protein 5 |
| AV002340 | RIKEN cDNA 4833442J19 gene |
| BB025325 | RIKEN cDNA 5330427M13 gene |
| BC002065 | serine (or cysteine) proteinase inhibitor, clade A, member 3G |
| NM_009217 | somatostatin receptor 2 |
| AK012130 | translocase of inner mitochondrial membrane 22 homolog (yeast) |
| BM239048 | Transcribed sequences |
| BB797041 | Transcribed sequence with weak similarity to protein ref: NP_081764.1(M. musculus) |
| | RIKEN cDNA 5730493B19 [Mus musculus] |
| C85885 | high mobility group box 2 |
| BB639333 | 3 days neonate thymus cDNA, RIKEN full-length enriched library, clone: A630090A15 |
| | product: unknown EST, full insert sequence |
| AW049828 | UI-M-BH1-anr-b-02-0-UI.s1 NIH_BMAP_M_S2 Mus musculus cDNA clone UI-M- |
| | BH1-anr-b-02-0-UI 3′, mRNA sequence. |
| NM_011641 | transformation related protein 63 |
| BB534983 | RIKEN cDNA A930027H06 gene |
| B0020014 | EH-domain containing 2 |
| BB530332 | BB530332 RIKEN full-length enriched, 0 day neonate lung Mus musculus cDNA |
| | clone E030009D19 3′, mRNA sequence. |
| BG229308 | porcollagen, type VIII, alpha 2 |
| NM_019388 | CD86 antigen |
| AK014814 | Mus musculus adult male testis cDNA, RIKEN full-length enriched library, |
| | clone: 4921504P05 product: unclassifiable, full insert sequence. |
| NM_013675 | spectrin beta 1 |
| BM221361 | CLIP associating protein 2 |
| BG069230 | Transcribed sequences |
| BF463236 | 16 days neonate cerebellum cDNA, RIKEN full-length enriched library, |
| | clone: 9630053P03 product: hypothetical protein, full insert sequence |
| AU043294 | Transcribed sequences |
| L20048 | interleukin 2 receptor, gamma chain |
| AI451513 | Transcribed sequences |
| AV231866 | UDP-N-acetyl-alpha-D-galactosamine:polypeptide N- |
| | acetylgalactosaminyltransferase 6 |
| NM_007646 | CD38 antigen |
| NM_008152 | G-protein coupled receptor 65 |
| BE635194 | Transcribed sequence with weak similarity to protein ref: NP_081764.1(M. musculus) |
| | RIKEN cDNA 5730493B19 [Mus musculus] |
| BG071110 | Similar to FLJ14075 protein (LOC217431), mRNA |
| AK004794 | MAM domain containing 2 |
| BE133829 | sequestosome 1 |
| NM_008141 | guanine nucleotide binding protein, alpha transducing 2 |
| AW519657 | RIKEN cDNA 6330407A03 gene |
| BB308375 | Transcribed sequence with weak similarity to protein ref: NP_058079.1(M. musculus) |
| | Ser/Arg-related nuclear matrix protein; plenty-of-prolines-101; serine/arginine |
| | repetitive matrix protein 1 [Mus musculus] |
| BB505010 | Transcribed sequences |
| BB149977 | RIKEN cDNA 9130013K24 gene |
| AV223474 | AV223474 RIKEN full-length enriched, 18 days pregnant, placenta and extra |
| | embryonic tissue Mus musculus cDNA clone 3830411J16 3′, mRNA sequence. |
| AK007024 | Adult male testis cDNA, RIKEN full-length enriched library, clone: 4930545O22 |
| | product: unknown EST, full insert sequence |
| BE955394 | RIKEN cDNA 2900052N01 gene |
| BE686864 | RIKEN cDNA 4932432N11 gene |
| BG141912 | RIKEN cDNA 8430417A20 gene |
| BG068105 | H3061G10-3 NIA Mouse 15K cDNA Clone Set Mus musculus cDNA clone H3061G10 |
| | 3′, mRNA sequence. |
| AF319542 | potassium channel, subfamily K, member 5 |
| BM230224 | RIKEN cDNA 2700049A03 gene |
| AV214133 | AV214133 RIKEN full-length enriched, ES cells Mus musculus cDNA clone |
| | 2410133J07 3′, mRNA sequence. |
| BB394986 | aquaporin 6 |
| NM_009709 | aryl hydrocarbon receptor nuclear translocator |
| BB075541 | Adult male diencephalon cDNA, RIKEN full-length enriched library, clone: 9330109G15 |
| | product: unknown EST, full insert sequence |
| BG147188 | Transcribed sequences |
| BB518380 | 16 days neonate heart cDNA, RIKEN full-length enriched library, clone: D830029C24 |
| | product: unknown EST, full insert sequence |
| AW492648 | Transcribed sequences |
| BC024358 | tropomyosin 2, beta |
| BB389192 | RIKEN cDNA 6030446N20 gene |
| AV240088 | fibroblast growth factor 5 |
| NM_009526 | wingless-related MMTV integration site 6 |
| AI606033 | formin binding protein 1 |
| BB333334 | kit oncogene |
| C77949 | Transcribed sequences |
| AK012253 | RIKEN cDNA 2700017A04 gene |
| AJ277212 | RIKEN cDNA 1110028G01 gene |
| BB085654 | BB085654 RIKEN full-length enriched, adult male diencephalon Mus musculus cDNA |
| | clone 9330200P05 3′, mRNA sequence. |
| BM123510 | Transcribed sequences |
| BB767243 | Transcribed sequences |
| NM_011746 | makorin, ring finger protein, 3 |
| BB493178 | 13 days embryo stomach cDNA, RIKEN full-length enriched library, |
| | clone: D530031M14 product: unknown EST, full insert sequence |
| BB380041 | cDNA sequence BC025526 |
| AK008705 | RIKEN cDNA 2210011C24 gene |
| AW556790 | RIKEN cDNA 4930520C08 gene |
| BG072861 | Transcribed sequences |
| AI449122 | Transcribed sequence with weak similarity to protein pir: I58401 (M. musculus) I58401 |
| | protein-tyrosine kinase |
| BB233192 | special AT-rich sequence binding protein 1 |
| AI931862 | biglycan |
| AK009828 | neuraminidase 2 |
| NM_030127 | RIKEN cDNA 9530081K03 gene |
| BB125332 | RIKEN cDNA 1300004C11 gene |
| AV229424 | procollagen, type V, alpha 2 |
| C78041 | C78041 Mouse 3.5-dpc blastocyst cDNA Mus musculus cDNA clone J0041H05 3′, |
| | mRNA sequence. |
| BB229853 | Transcribed sequences |
| BM244351 | tripartite motif protein 12 |
| BB366425 | BB366425 RIKEN full-length enriched, 16 days embryo head Mus musculus cDNA |
| | clone C130033K03 3′, mRNA sequence. |
| AK017409 | Mus musculus 6 days neonate head cDNA, RIKEN full-length enriched library, |
| | clone: 5430439A09 product: transcription factor AP-2, alpha, full insert sequence. |
| AK020680 | 16 days neonate cerebellum cDNA, RIKEN full-length enriched library, |
| | clone: 9630015K15 product: unclassifiable, full insert sequence |
| BM208085 | Hypothetical gene supported by BC031136 (LOC381297), mRNA |
| BB197238 | RIKEN cDNA 1600031M04 gene |
| BB320816 | Transcribed sequence |
| BB726544 | DMRT-like family B with proline-rich C-terminal, 1 |
| BF134369 | RIKEN cDNA 2810443J12 gene |
| NM_023517 | tumor necrosis factor (ligand) superfamily, member 13 |
| BB771139 | expressed sequence AU020939 |
| BG073146 | RIKEN cDNA 6030408C04 gene |
| BC015253 | arachidonate 15-lipoxygenase, second type |
| C85065 | RIKEN cDNA 9130410M22 gene |
| BE992189 | phosphatidylinositol glycan, class M |
| BB193866 | Transcribed sequences |
| BG070848 | oxysterol binding protein-like 6 |
| AA414993 | vc50a06.r1 Knowles Solter mouse 2 cell Mus musculus cDNA clone IMAGE: 777970 |
| | 3′, mRNA sequence. |
| BQ033138 | 2′-5′ oligoadenylate synthetase-like 2 |
| NM_020008 | C-type (calcium dependent, carbohydrate recognition domain) lectin, superfamily |
| | member 12 |
| AV321065 | RIKEN cDNA 1110051B16 gene |
| BM508503 | RIKEN cDNA 1110039B18 gene |
| BF021309 | RIKEN cDNA 2700091H24 gene |
| BB485193 | 13 days embryo lung cDNA, RIKEN full-length enriched library, clone: D430030G11 |
| | product: unknown EST, full insert sequence |
| BC018418 | 6-phosphofructo-2-kinase/fructose-2,6-biphosphatase 2 |
| BB157298 | metastasis suppressor 1 |
| BB524087 | imprinted and ancient |
| AU024680 | beta-transducin repeat containing protein |
| AW475993 | expressed sequence AI256711 |
| NM_053190 | endothelial differentiation, sphingolipid G-protein-coupled receptor, 8 |
| NM_020033 | ankyrin repeat domain 2 (stretch responsive muscle) |
| NM_028071 | coactosin-like 1 (Dictyostelium) |
| BM231135 | Transcribed sequences |
| BG068678 | RIKEN cDNA 9130011B11 gene |
| BB463474 | 12 days embryo spinal ganglion cDNA, RIKEN full-length enriched library, |
| | clone: D130080L18 product: unclassifiable, full insert sequence |
| AK015795 | RIKEN cDNA 4930515G13 gene |
| NM_028238 | Rab38, member of RAS oncogene family |
| BB529038 | 15 days embryo head cDNA, RIKEN full-length enriched library, clone: D930049J19 |
| | product: unknown EST, full insert sequence |
| AK020278 | Similar to Jun dimerization protein 1 gene (LOC381319), mRNA |
| AK010621 | RIKEN cDNA 2410030J07 gene |
| BB551060 | 2 days pregnant adult female oviduct cDNA, RIKEN full-length enriched library, |
| | clone: E230025M10 product: unknown EST, full insert sequence |
| BB756069 | tissue factor pathway inhibitor |
| AK018322 | RIKEN cDNA 6530404N21 gene |
| BG066235 | Transcribed sequences |
| BB160137 | Transcribed sequence with strong similarity to protein sp: P00722 (E. coli) |
| | BGAL_ECOLI Beta-galactosidase |
| BF319989 | phospholipid scramblase 1 |
| BB245809 | fragile X mental retardation 2 homolog |
| BB121269 | BB121269 RIKEN full-length enriched, adult male urinary bladder Mus musculus |
| | cDNA clone 9530081E16 3′, mRNA sequence. |
| AW987375 | protein tyrosine phosphatase, non-receptor type 21 |
| AV361868 | AV361868 RIKEN full-length enriched, adult male corpus striatum Mus musculus |
| | cDNA clone 7630402M06 3′, mRNA sequence. |
| BM507943 | growth arrest specific 2 |
| BG085035 | 10 days lactation, adult female mammary gland cDNA, RIKEN full-length enriched |
| | library, clone: D730048J04 product: unknown EST, full insert sequence |
| BB452660 | 15 days embryo head cDNA, RIKEN full-length enriched library, clone: D930017N16 |
| | product: hypothetical protein, full insert sequence |
| C79043 | Transcribed sequences |
| AV370609 | hypothetical protein 9030224M15 |
| AK019695 | RIKEN cDNA 4930524L23 gene |
| AV230021 | Transcribed sequences |
| BG065720 | Transcribed sequences |
| AK012899 | Hypothetical LOC229146 (LOC229146), mRNA |
| BC022224 | cDNA sequence BC022224 |
| AV333667 | estrogen related receptor, beta |
| BG063116 | Transcribed sequences |
| AK012302 | carbonic anhydrase 10 |
| BB459917 | RIKEN cDNA B230118H07 gene |
| AV382118 | ATP-binding cassette, sub-family B (MDR/TAP), member 10 |
| BB220605 | Adult male aorta and vein cDNA, RIKEN full-length enriched library, |
| | clone: A530060O18 product: unknown EST, full insert sequence |
| BB707145 | BB707145 RIKEN full-length enriched, in vitro fertilized eggs Mus musculus cDNA |
| | clone 7420497J01 3′, mRNA sequence. |
| AK010725 | RIKEN cDNA 2410076I21 gene |
| NM_010181 | fibrillin 2 |
| BB234940 | discoidin domain receptor family, member 1 |
| BF730667 | Transcribed sequence with moderate similarity to protein pir: S12207 (M. musculus) |
| | S12207 hypothetical protein |
| BF585144 | 602101888F2 NCI_CGAP_Co24 Mus musculus cDNA clone IMAGE: 4225388 5′, mRNA |
| | sequence. |
| AV381193 | RIKEN cDNA A430065P19 gene |
| BB386853 | 0 day neonate cerebellum cDNA, RIKEN full-length enriched library, |
| | clone: C230048M01 product: unclassifiable, full insert sequence |
| AU015122 | Transcribed sequence with weak similarity to protein ref: NP_079268.1 (H. sapiens) |
| | hypothetical protein FLJ12547 [Homo sapiens] |
| AI451392 | RIKEN cDNA 2010001J22 gene |
| BB139669 | Transcribed sequences |
| AK005608 | Adult male testis cDNA, RIKEN full-length enriched library, clone: 1700001L05 |
| | product: unknown EST, full insert sequence |
| BE956926 | galactosidase, beta 1 |
| BB040432 | RIKEN cDNA 9130023H24 gene |
| BG075061 | Transcribed sequences |
| AW123502 | Transcribed sequences |
| AV280494 | RIKEN cDNA 4933428A15 gene |
| AV156640 | AV156640 Mus musculus head C57BL/6J 12-day embryo Mus musculus cDNA |
| | clone 3000003N22, mRNA sequence. |
| BG067081 | DNA segment, Chr 19, ERATO Doi 386, expressed |
| BB524087 | imprinted and ancient |
| BC016497 | eukaryotic translation initiation factor 2, subunit 1 alpha |
| BE685667 | Transcribed sequences |
| BB100748 | BB100748 RIKEN full-length enriched, 12 days embryo, embryonic body between |
| | diaphragm region and neck Mus musculus cDNA clone 9430074A13 3′, mRNA |
| | sequence. |
| BB380053 | BB380053 RIKEN full-length enriched, 0 day neonate cerebellum Mus musculus |
| | cDNA clone C230003J04 3′, mRNA sequence. |
| AV324454 | AV324454 RIKEN full-length enriched, 11 days embryo head Mus musculus cDNA |
| | clone 6230424H17 3′, mRNA sequence. |
| BB027903 | adaptor-related protein complex 3, delta subunit |
| AK016329 | RIKEN cDNA 4930579K19 gene |
| AA415004 | phosphatidylinositol 3-kinase, C2 domain containing, alpha polypeptide |
| BB133120 | BB133120 RIKEN full-length enriched, adult male bone Mus musculus cDNA clone |
| | 9830005L10 3′ similar to X60785 Cricetulus griseus (chinese hamster) mRNA for |
| | beta tubulin (clone B3T), mRNA sequence. |
| AW493518 | Clone IMAGE: 1514139, mRNA |
| AK003824 | RIKEN cDNA 1110019K23 gene |
| AK020441 | 12 days embryo embryonic body between diaphragm region and neck cDNA, RIKEN |
| | full-length enriched library, clone: 9430027B09 product: unclassifiable, full insert |
| | sequence |
| BB496114 | RIKEN cDNA 5730446D14 gene |
| BE628614 | annexin A4 |
| BC013480 | cystathionine beta-synthase |
| BB526903 | Transcribed sequences |
| BB125476 | RIKEN cDNA 5330427D05 gene |
| BG066735 | Similar to hepatitis A virus cellular receptor 1; T-cell immunoglobulin and mucin |
| | domain containing 1 (LOC210535), mRNA |
| NM_011579 | T-cell specific GTPase |
| BB091194 | Adult male thymus cDNA, RIKEN full-length enriched library, clone: 5832446B02 |
| | product: unclassifiable, full insert sequence |
| NM_031374 | testis expressed gene 15 |
| BB366710 | Transcribed sequences |
| AI853240 | RIKEN cDNA D230016N13 gene |
| AV254043 | Transcribed sequences |
| BB324785 | Transcribed sequences |
| AK011411 | RIKEN cDNA 2610016D08 gene |
| BB277828 | Adult retina cDNA, RIKEN full-length enriched library, clone: A930007K23 |
| | product: unclassifiable, full insert sequence |
| BI648497 | angiotensin II, type I receptor-associated protein |
| BG079145 | H3036D01-5 NIA Mouse 15K cDNA Clone Set Mus musculus cDNA clone H3036D01 |
| | 5′, mRNA sequence. |
| BB139866 | RIKEN cDNA 9830166K06 gene |
| BB447914 | zinc finger protein 503 |
| BM234447 | Transcribed sequences |
| AV330315 | solute carrier family 22 (organic anion transporter), member 8 |
| NM_016756 | cyclin-dependent kinase 2 |
| BG067899 | Transcribed sequences |
| AK017558 | Mus musculus 8 days embryo whole body cDNA, RIKEN full-length enriched library, |
| | clone: 5730414A19 product: multiple inositol polyphosphate histidine phosphatase 1, |
| | full insert sequence. |
| AK016629 | RIKEN cDNA 4933414E04 gene |
| BG066725 | myosin IB |
| AK003675 | Mus musculus 18-day embryo whole body cDNA, RIKEN full-length enriched library, |
| | clone: 1110014B11 product: similar to C316G12.2 (NOVEL PROTEIN SIMILAR TO |
| | PREDICTED YEAST, WORM AND ARCHAE-BACTERIAL PROTEINS) (SIMILAR TO |
| | UND313) (S. CERVISIAE) (HYPOTHETICAL 33.6 KDA PROTEIN) [Homo sapiens], |
| | full insert sequence. |
| AY083458 | CD109 antigen |
| BB110572 | Transcribed sequences |
| AW987495 | hypothetical protein 9130207N01 |
| BG070025 | Transcribed sequences |
| AK018513 | 16 days neonate thymus cDNA, RIKEN full-length enriched library, |
| | clone: A130034L04 product: unknown EST, full insert sequence |
| BB126186 | 16 days neonate cerebellum cDNA, RIKEN full-length enriched library, |
| | clone: 9630012D08 product: unknown EST, full insert sequence |
| AV312787 | AV312787 RIKEN full-length enriched, adult male thymus Mus musculus cDNA clone |
| | 5830405J08 3′, mRNA sequence. |
| AA221216 | RIKEN cDNA C130046K22 gene |
| BB408396 | RIKEN cDNA 6230410P16 gene |
| AV069898 | AV069898 Mus musculus small intestine C57BL/6J adult Mus musculus cDNA clone |
| | 2010313H18, mRNA sequence. |
| BB238385 | Transcribed sequences |
| BG069965 | Transcribed sequences |
| AV320664 | RIKEN cDNA E130309F12 gene |
| BB254163 | CD47 antigen (Rh-related antigen, integrin-associated signal transducer) |
| AW259474 | immunoglobulin mu binding protein 2 |
| BB392923 | gap junction membrane channel protein alpha 9 |
| AA690806 | serologically defined colon cancer antigen 8 |
| AV327883 | Transcribed sequences |
| AK016729 | 6-phosphofructo-2-kinase/fructose-2,6-biphosphatase 2 |
| AK017151 | 11 days pregnant adult female ovary and uterus cDNA, RIKEN full-length enriched |
| | library, clone: 5033404E19 product: cortactin binding protein 2, full insert sequence |
| AI595843 | Transcribed sequences |
| AI036317 | loricrin |
| L43371 | phosphatidic acid phosphatase 2a |
| BB336256 | Transcribed sequence with moderate similarity to protein sp: P00722 (E. coli) |
| | BGAL_ECOLI Beta-galactosidase |
| BB378700 | BB378700 RIKEN full-length enriched, 16 days embryo head Mus musculus cDNA |
| | clone C130095G05 3′ similar to AF026259 Mus musculus receptor-like tyrosine |
| | kinase (Nep) mRNA, mRNA sequence. |
| AV174487 | 18-day embryo whole body cDNA, RIKEN full-length enriched library, |
| | clone: 1110017D07 product: unclassifiable, full insert sequence |
| BB109562 | RIKEN cDNA 4732435N03 gene |
| BC027165 | centaurin, alpha 2 |
| BM213197 | casein kinase 1, epsilon |
| BB099487 | minichromosome maintenance deficient 6 (MIS5 homolog, S. pombe) (S. cerevisiae) |
| NM_008184 | glutathione S-transferase, mu 6 |
| BB407699 | RIKEN cDNA C430002N04 gene |
| BB124954 | chemokine (C—C motif) receptor 9 |
| AF402613 | FYVE, RhoGEF and PH domain containing 4 |
| BB283168 | Transcribed sequences |
| BG075006 | 15 days embryo head cDNA, RIKEN full-length enriched library, clone: D930049J19 |
| | product: unknown EST, full insert sequence |
| BB469236 | 12 days embryo eyeball cDNA, RIKEN full-length enriched library, clone: D230023K22 |
| | product: unclassifiable, full insert sequence |
| BG092211 | 3 days neonate thymus cDNA, RIKEN full-length enriched library, clone: A630021E21 |
| | product: unknown EST, full insert sequence |
| BB154331 | CD8 antigen, alpha chain |
| NM_053216 | RIKEN cDNA 1700102P08 gene |
| BI687857 | lung carcinoma myc related oncogene 1 |
| AV293250 | Transcribed sequence with weak similarity to protein pir: S70642 (R. norvegicus) |
| | S70642 ubiquitin ligase Nedd4 - rat |
| AK015932 | Adult male testis cDNA, RIKEN full-length enriched library, clone: 4930567J20 |
| | product: unclassifiable, full insert sequence |
| BB354676 | RIKEN cDNA C030001A19 gene |
| NM_030709 | transmembrane protease, serine 5 (spinesin) |
| NM_133244 | pleckstrin homology domain containing, family K member 1 |
| NM_053134 | protocadherin beta 9 |
| BB319063 | BB319063 RIKEN full-length enriched, adult male corpora quadrigemina Mus |
| | musculus cDNA clone B230378I04 3′, mRNA sequence. |
| BE981788 | zinc finger protein 533 |
| BM117404 | Transcribed sequences |
| U08020 | procollagen, type I, alpha 1 |
| BB451832 | neurofibromatosis 1 |
| AK017243 | 6 days neonate head cDNA, RIKEN full-length enriched library, clone: 5430400D12 |
| | product: unknown EST, full insert sequence |
| BG066572 | Transcribed sequences |
| BE335894 | 11 days embryo whole body cDNA, RIKEN full-length enriched library, |
| | clone: 2700029L08 product: unknown EST, full insert sequence |
| AW125726 | zinc finger, MYND domain containing 19 |
| BB333971 | 10 days neonate medulla oblongata cDNA, RIKEN full-length enriched library, |
| | clone: B830017H08 product: hypothetical protein, full insert sequence |
| BI151331 | 3 days neonate thymus cDNA, RIKEN full-length enriched library, clone: A630040D01 |
| | product: unknown EST, full insert sequence |
| BB471757 | Transcribed sequences |
| BB114398 | BB114398 RIKEN full-length enriched, adult male urinary bladder Mus musculus |
| | cDNA clone 9530045O15 3′ similar to U57362 Rattus norvegicus collagen XII alpha 1 |
| | (Col12a1) mRNA, mRNA sequence. |
| AK019180 | RIKEN cDNA 2610311B01 gene |
| NM_016793 | zinc finger protein 98 |
| AK018247 | Adult male medulla oblongata cDNA, RIKEN full-length enriched library, |
| | clone: 6330571C24 product: unclassifiable, full insert sequence |
| BG075349 | Transcribed sequences |
| L06502 | paired related homeobox 1 |
| BF661746 | Transcribed sequence with weak similarity to protein sp: P11369 (M. musculus) |
| | POL2_MOUSE Retrovirus-related POL polyprotein [Contains: Reverse transcriptase; |
| | Endonuclease] |
| AI413999 | ma78b02.x1 Soares mouse p3NMF19.5 Mus musculus cDNA clone IMAGE: 316779 3′, |
| | mRNA sequence. |
| BB203988 | 0 day neonate thymus cDNA, RIKEN full-length enriched library, clone: A430055L04 |
| | product: unclassifiable, full insert sequence |
| NM_008331 | interferon-induced protein with tetratricopeptide repeats 1 |
| BB147678 | dual oxidase 2 |
| BG230324 | RIKEN cDNA A330063D19 gene |
| BE633038 | RIKEN cDNA 2310032F03 gene |
| AI785192 | mu16h10.y1 Soares_thymus_2NbMT Mus musculus cDNA clone IMAGE: 639619 5′, |
| | mRNA sequence. |
| BB020616 | BB020616 RIKEN full-length enriched, adult male pituitary gland Mus musculus |
| | cDNA clone 5330402N14 3′, mRNA sequence. |
| BB359162 | BB359162 RIKEN full-length enriched, adult male corpus striatum Mus musculus |
| | cDNA clone C030036G09 3′ similar to AF131753 Homo sapiens clone 24859, mRNA |
| | sequence. |
| AK019583 | RIKEN cDNA 4930425F17 gene |
| BC020004 | G protein-coupled receptor, family C, group 5, member B |
| BB541632 | cDNA sequence BC051083 |
| AV272484 | ubiquitin specific protease 20 |
| AV337827 | Transcribed sequences |
| BG073178 | Adult male thymus cDNA, RIKEN full-length enriched library, clone: 5830453E13 |
| | product: unknown EST, full insert sequence |
| BF165671 | 601777979F1 NCI_CGAP_Lu29 Mus musculus cDNA clone IMAGE: 4019538 5′, mRNA |
| | sequence. |
| AK002481 | RIKEN cDNA 0610010I15 gene |
| AK015254 | RIKEN cDNA 4930430K04 gene |
| BB319311 | Transcribed sequences |
| AU024158 | Transcribed sequences |
| BB136012 | capping protein (actin filament), gelsolin-like |
| BB373326 | RIKEN cDNA 2810438M17 gene |
| BB361377 | Transcribed sequences |
| NM_031182 | transcription factor AP-4 (activating enhancer-binding protein 4) |
| BB759556 | myoneurin |
| BC010305 | gene rich cluster, C9 gene |
| L06502 | paired related homeobox 1 |
| AW553120 | Transcribed sequences |
| NM_009791 | calmodulin binding protein 1 |
| BB308198 | RIKEN cDNA A930014I12 gene |
| BG069991 | H3083D07-3 NIA Mouse 15K cDNA Clone Set Mus musculus cDNA clone H3083D07 |
| | 3′, mRNA sequence. |
| BE948877 | DNA segment, Chr 18, ERATO Doi 653, expressed |
| C79125 | RIKEN cDNA 2010002H18 gene |
| BM239721 | cDNA sequence BC019206 |
| AW494906 | RIKEN cDNA 1110006I15 gene |
| BB325847 | tripartite motif protein 24 |
| AV326297 | expressed sequence AI894139 |
| BG065751 | Transcribed sequences |
| BM124366 | leptin receptor |
| BG069831 | Transcribed sequences |
| BC010202 | Kirsten rat sarcoma oncogene 2, expressed |
| NM_021712 | solute carrier family 18 (vesicular monoamine), member 3 |
| BB443362 | BB443362 RIKEN full-length enriched, 9 days embryo Mus musculus cDNA clone |
| | D030040N11 3′, mRNA sequence. |
| BB297543 | Transcribed sequence with weak similarity to protein pir: S65824 (H. sapiens) S65824 |
| | reverse transcriptase homolog - human transposon L1.1 |
| BB522422 | Transcribed sequence with weak similarity to protein sp: P15056 (H. sapiens) |
| | KRAB_HUMAN B-Raf proto-oncogene serine/threonine-protein kinase |
| NM_008998 | RAB17, member RAS oncogene family |
| BB153779 | 0 day neonate thymus cDNA, RIKEN full-length enriched library, clone: A430023L13 |
| | product: unknown EST, full insert sequence |
| AA185884 | Adult male aorta and vein cDNA, RIKEN full-length enriched library, |
| | clone: A530055P18 product: unknown EST, full insert sequence |
| AI481031 | RIKEN cDNA 4631416I11 gene |
| BM877552 | Transcribed sequence with weak similarity to protein ref: NP_081764.1 (M. musculus) |
| | RIKEN cDNA 5730493B19 [Mus musculus] |
| BG071073 | Transcribed sequences |
| AV272776 | Transcribed sequences |
| BB078011 | Adult male diencephalon cDNA, RIKEN full-length enriched library, clone: 9330150G21 |
| | product: unknown EST, full insert sequence |
| BB656631 | SRY-box containing gene 11 |
| BM196689 | Transcribed sequences |
| AV245881 | AV245881 RIKEN full-length enriched, 0 day neonate head Mus musculus cDNA |
| | clone 4832402K11 3′, mRNA sequence. |
| AK020698 | RIKEN cDNA A030006P16 gene |
| BB656539 | BB656539 RIKEN full-length enriched, 12 days embryo spinal ganglion Mus musculus |
| | cDNA clone D130036O06 5′, mRNA sequence. |
| BB253461 | UBX domain containing 2 |
| BB012661 | Adult male testis cDNA, RIKEN full-length enriched library, clone: 4930452E19 |
| | product: unclassifiable, full insert sequence |
| AW554436 | acid phosphatase 1, soluble |
| BB155176 | 16 days neonate thymus cDNA, RIKEN full-length enriched library, |
| | clone: A130023P20 product: unclassifiable, full insert sequence |
| NM_016907 | serine protease inhibitor, Kunitz type 1 |
| BB735978 | BB735978 RIKEN full-length enriched, 6 days neonate spleen Mus musculus cDNA |
| | clone F430006D24 3′, mRNA sequence. |
| AK021082 | Adult male corpus striatum cDNA, RIKEN full-length enriched library, |
| | clone: C030014O09 product: unclassifiable, full insert sequence |
| AV258920 | AV258920 RIKEN full-length enriched, adult male testis (DH10B) Mus musculus |
| | cDNA clone 4930402J02 3′, mRNA sequence. |
| BB207363 | 0 day neonate thymus cDNA, RIKEN full-length enriched library, clone: A430081G06 |
| | product: unknown EST, full insert sequence |
| BB204671 | Transcribed sequences |
| BM249924 | 10 days neonate skin cDNA, RIKEN full-length enriched library, clone: 4733401A01 |
| | product: unknown EST, full insert sequence |
| BB394986 | aquaporin 6 |
| AV166268 | RIKEN cDNA 3110043A19 gene |
| AV044909 | AV044909 Mus musculus adult C57BL/6J testis Mus musculus cDNA clone |
| | 1700040E03, mRNA sequence. |
| BM243794 | K0702A03-3 NIA Mouse Hematopoietic Stem Cell (Lin-/c-Kit-/Sca-1-) cDNA |
| | Library (Long) Mus musculus cDNA clone K0702A03 3′, mRNA sequence. |
| AK013119 | Mus musculus 10, 11 days embryo whole body cDNA, RIKEN full-length enriched |
| | library, clone: 2810420M18 product: SIMILAR TO CHROMOSOME CONDENSATION |
| | 1-LIKE homolog [Mus musculus], full insert sequence. |
| AV344962 | AV344962 RIKEN full-length enriched, adult male olfactory bulb Mus musculus cDNA |
| | clone 6430548L14 3′, mRNA sequence. |
| BB450549 | kinesin family member 6 |
| AK018014 | Mus musculus adult male thymus cDNA, RIKEN full-length enriched library, |
| | clone: 5830455I16 product: T-cell receptor beta chain homolog [Mus musculus], full |
| | insert sequence. |
| NM_011832 | insulin receptor-related receptor |
| BB286270 | BB286270 RIKEN full-length enriched, 2 cells egg Mus musculus cDNA clone |
| | B020011L17 3′ similar to U31668 Rattus norvegicus transcription factor E2F-5 |
| | mRNA, mRNA sequence. |
| NM_007626 | chromobox homolog 5 (Drosophila HP1a) |
| AV373598 | RIKEN cDNA 9030624O13 gene |
| AJ245857 | carbonic anhydrase 9 |
| AY015061 | large tumor suppressor 2 |
| BB150699 | Transcribed sequences |
| AK017228 | Adult male xiphoid cartilage cDNA, RIKEN full-length enriched library, |
| | clone: 5230400M06 product: unclassifiable, full insert sequence |
| BG068519 | Adult male testis cDNA, RIKEN full-length enriched library, clone: 1700001I24 |
| | product: unknown EST, full insert sequence |
| AA409749 | EST01534 Mouse 7.5 dpc embryo ectoplacental cone cDNA library Mus musculus |
| | cDNA clone C0011A07 3′, mRNA sequence. |
| NM_026084 | RIKEN cDNA 3110070M22 gene |
| BB139156 | Similar to KIAA1529 protein (LOC269531), mRNA |
| AI449585 | UDP-glucose ceramide glucosyltransferase |
| BB026232 | BB026232 RIKEN full-length enriched, adult male pituitary gland Mus musculus |
| | cDNA clone 5330432C16 3′, mRNA sequence. |
| AK020257 | Mus musculus adult male colon cDNA, RIKEN full-length enriched library, |
| | clone: 9030617G22 product: inversin, full insert sequence. |
| AW491934 | Adult male testis cDNA, RIKEN full-length enriched library, clone: 1700063D05 |
| | product: unknown EST, full insert sequence |
| BB642740 | embryonic ectoderm development |
| AF220015 | tripartite motif protein 30 |
| NM_015802 | deleted in liver cancer 1 |
| BF318372 | Transcribed sequences |
| NM_134256 | |
| BM241237 | H3 histone, family 3B |
| NM_133762 | RIKEN cDNA 5830426I05 gene |
| AK015753 | RIKEN cDNA 4930511H01 gene |
| BI328541 | kinesin family member 5B |
| BB480256 | BB480256 RIKEN full-length enriched, 13 days embryo heart Mus musculus cDNA |
| | clone D330045D12 3′, mRNA sequence. |
| BB324481 | prostaglandin I2 (prostacyclin) synthase |
| AI839700 | UI-M-AN0-acp-f-04-0-UI.s1 NIH_BMAP_MBG Mus musculus cDNA clone UI-M- |
| | AN0-acp-f-04-0-UI 3′, mRNA sequence. |
| NM_011612 | tumor necrosis factor receptor superfamily, member 9 |
| BC025020 | RIKEN cDNA 2810049G06 gene |
| AK009476 | RIKEN cDNA 2310022K01 gene |
| BB740218 | coronin, actin binding protein 1A |
| BE951016 | Transcribed sequences |
| BB283960 | Transcribed sequences |
| AK019582 | RIKEN cDNA 1700020F09 gene |
| AI120134 | uh82g05.r1 Soares mouse urogenital ridge NMUR Mus musculus cDNA clone |
| | IMAGE: 1764248 5′, mRNA sequence. |
| BF463223 | RIKEN cDNA 4930473B18 gene |
| NM_028602 | testis expressed gene 19 |
| BC022676 | RIKEN cDNA 4933433D23 gene |
| AW123715 | Transcribed sequence with strong similarity to protein sp: P00722 (E. coli) |
| | BGAL_ECOLI Beta-galactosidase |
| BB746538 | Adult male epididymis cDNA, RIKEN full-length enriched library, clone: 9230111E07 |
| | product: unknown EST, full insert sequence |
| BE981626 | Transcribed sequences |
| BB519063 | 16 days neonate heart cDNA, RIKEN full-length enriched library, clone: D830033F17 |
| | product: unknown EST, full insert sequence |
| AI414330 | salvador homolog 1 (Drosophila) |
| BC011063 | homeo box A5 |
| NM_033607 | ubiquitin carboxyl-terminal esterase L4 |
| BG229036 | Transcribed sequence with weak similarity to protein ref: NP_081764.1 (M. musculus) |
| | RIKEN cDNA 5730493B19 [Mus musculus] |
| BG075036 | H3142E05-3 NIA Mouse 15K cDNA Clone Set Mus musculus cDNA clone H3142E05 |
| | 3′, mRNA sequence. |
| BG072319 | RIKEN cDNA 5830484A20 gene |
| AK016584 | RIKEN cDNA 1700007A21 gene |
| BB049129 | Transcribed sequences |
| BG069763 | Transcribed sequences |
| NM_023729 | germ cell-specific ankyrin, SAM and basic leucine zipper domain containing protein |
| BB709101 | BB709101 RIKEN full-length enriched, adult retina Mus musculus cDNA clone |
| | A930101K18 3′, mRNA sequence. |
| BB181225 | Adult male hypothalamus cDNA, RIKEN full-length enriched library, |
| | clone: A230089O20 product: potassium voltage-gated channel, subfamily Q, member |
| | 5, full insert sequence |
| BM199862 | CCCTC-binding factor |
| BB159664 | eukaryotic translation initiation factor 4, gamma 2 |
| BF019839 | AKT1 substrate 1 (proline-rich) |
| AW546412 | Transcribed sequences |
| AW555749 | Transcribed sequences |
| AK006425 | RIKEN cDNA 4933425L11 gene |
| NM_008567 | minichromosome maintenance deficient 6 (MIS5 homolog, S. pombe) (S. cerevisiae) |
| BG069641 | H3078E08-3 NIA Mouse 15K cDNA Clone Set Mus musculus cDNA clone H3078E08 |
| | 3′, mRNA sequence. |
| BB549860 | 2 days pregnant adult female oviduct cDNA, RIKEN full-length enriched library, |
| | clone: E230017O17 product: unknown EST, full insert sequence |
| AV244075 | 15 days embryo head cDNA, RIKEN full-length enriched library, clone: D930037F10 |
| | product: unknown EST, full insert sequence |
| AV117956 | AV117956 Mus musculus C57BL/6J 10-day embryo Mus musculus cDNA clone |
| | 2610206D24, mRNA sequence. |
| BG230349 | Transcribed sequence with moderate similarity to protein pir: S12207 (M. musculus) |
| | S12207 hypothetical protein |
| AA739023 | vv66a11.r1 Stratagene mouse skin (#937313) Mus musculus cDNA clone |
| | IMAGE: 1227356 5′, mRNA sequence. |
| AI481208 | engulfment and cell motility 3, ced-12 homolog (C. elegans) |
| BQ129058 | jumonji, AT rich interactive domain 1A (Rbp2 like) |
| AK018665 | Adult male cecum cDNA, RIKEN full-length enriched library, clone: 9130409J20 |
| | product: unclassifiable, full insert sequence |
| BC003985 | RIKEN cDNA 9130003O20 gene |
| BM247816 | Transcribed sequence with moderate similarity to protein ref: NP_081764.1 |
| | (M. musculus) RIKEN cDNA 5730493B19 [Mus musculus] |
| NM_026152 | RIKEN cDNA O610010D20 gene |
| BB443857 | RIKEN cDNA 3010021M21 gene |
| AV226646 | Transcribed sequence with moderate similarity to protein pir: S14234 (M. musculus) |
| | S14234 hypothetical protein - mouse |
| AK014780 | RIKEN cDNA 4833427G06 gene |
| AI596401 | phosphatidylserine synthase 2 |
| NM_008843 | prolactin induced protein |
| BB762627 | Transcribed sequences |
| BM942825 | Transcribed sequence with strong similarity to protein sp: P00722 (E. coli) |
| | BGAL_ECOLI Beta-galactosidase |
| NM_011387 | solute carrier family 10 (sodium/bile acid cotransporter family), member 1 |
| NM_010016 | decay accelerating factor 1 |
| AK020986 | Adult male corpora quadrigemina cDNA, RIKEN full-length enriched library, |
| | clone: B230204H03 product: unknown EST, full insert sequence |
| BC024104 | protein Z, vitamin K-dependent plasma glycoprotein |
| BG068591 | H3067B12-3 NIA Mouse 15K cDNA Clone Set Mus musculus cDNA clone H3067B12 |
| | 3′, mRNA sequence. |
| BB745263 | 10 day old male pancreas cDNA, RIKEN full-length enriched library, |
| | clone: 1810034E14 product: unknown EST, full insert sequence |
| BB637410 | Adult male aorta and vein cDNA, RIKEN full-length enriched library, |
| | clone: A530074N20 product: unknown EST, full insert sequence |
| AI845410 | carnitine deficiency-associated gene expressed in ventricle 3 |
| BB741897 | RIKEN cDNA 2510004L01 gene |
| BB485732 | 16 days embryo head cDNA, RIKEN full-length enriched library, clone: C130097K22 |
| | product: unknown EST, full insert sequence |
| BG066900 | Transcribed sequences |
| AK010391 | RIKEN cDNA 2410004I17 gene |
| BB211820 | Adult male corpora quadrigemina cDNA, RIKEN full-length enriched library, |
| | clone: B230317P14 product: unclassifiable, full insert sequence |
| BB087946 | RAS-related C3 botulinum substrate 1 |
| BB281055 | ceramide kinase-like |
| AW556975 | basic transcription factor 3 |
| BG067880 | H3059C01-3 NIA Mouse 15K cDNA Clone Set Mus musculus cDNA clone H3059C01 |
| | 3′, mRNA sequence. |
| AV290119 | Transcribed sequences |
| NM_023617 | RIKEN cDNA 1200011D03 gene |
| NM_031385 | testis expressed gene 18 |
| BG071947 | H3105A04-3 NIA Mouse 15K cDNA Clone Set Mus musculus cDNA clone H3105A04 |
| | 3′, mRNA sequence. |
| BI465746 | Transcribed sequences |
| AU046270 | AU046270 Mouse sixteen-cell-embryo cDNA Mus musculus cDNA clone J0950D05 |
| | 3′, mRNA sequence. |
| AV305128 | RIKEN cDNA A730012O14 gene |
| BG970109 | laminin B1 subunit 1 |
| AK015656 | protein kinase C, alpha binding protein |
| BB121627 | BB121627 RIKEN full-length enriched, adult male urinary bladder Mus musculus |
| | cDNA clone 9530082N06 3′, mRNA sequence. |
| C77776 | Transcribed sequences |
| BB738626 | ubiquitin-activating enzyme E1, Chr X |
| AK011140 | unnamed protein product; CDNA FLJ10805 FIS, CLONE NT2RP4000839, WEAKLY |
| | SIMILAR TO VEGETATIBLE INCOMPATIBILITY PROTEIN HET-E-1 homolog [Homo |
| | sapiens] (SPTR\|Q9NVD1, evidence: FASTY, 99.6% ID, 100% length, match = 1540) |
| | putative; Mus musculus 10 days embryo whole body cDNA, RIKEN full-length |
| | enriched library, clone: 2600001O03 product: CDNA FLJ10805 FIS, CLONE |
| | NT2RP4000839, WEAKLY SIMILAR TO VEGETATIBLE INCOMPATIBILITY PROTEIN |
| | HET-E-1 homolog [Homo sapiens], full insert sequence. |
| BB088759 | Transcribed sequences |
| AW551705 | Transcribed sequences |
| NM_025453 | RIKEN cDNA 1810018L02 gene |
| AV154314 | AV154314 Mus musculus hippocampus C57BL/6J adult Mus musculus cDNA clone |
| | 2900060N12, mRNA sequence. |
| BB807192 | kelch-like 5 (Drosophila) |
| AV331703 | Adult male medulla oblongata cDNA, RIKEN full-length enriched library, |
| | clone: 6330525124 product: unknown EST, full insert sequence |
| BB477765 | Transcribed sequences |
| BB541054 | Transcribed sequences |
| AK015641 | unnamed protein product; CAMP-RESPONSIVE ELEMENT MODULATOR homolog |
| | [Mus musculus] (SWISSPROT\|P27699, evidence: FASTY, 99% ID, 89.1% length, |
| | match = 911) putative; Mus musculus adult male testis cDNA, RIKEN full-length |
| | enriched library, clone: 4930488A05 product: CAMP-RESPONSIVE ELEMENT |
| | MODULATOR homolog [Mus musculus], full insert sequence. |
| BB435780 | Transcribed sequences |
| BB811070 | Transcribed sequences |
| BB228490 | Similar to Cca3 protein, clone IMAGE: 4506844, mRNA |
| AI482454 | vg40d04.x1 Soares_mammary_gland_NbMMG Mus musculus cDNA clone |
| | IMAGE: 863815 3′, mRNA sequence. |
| NM_009571 | zinc finger protein 1, Y linked |
| AW542416 | RIKEN cDNA 3830422N12 gene |
| X67278 | CEA-related cell adhesion molecule 1 |
| BB007136 | RIKEN cDNA 4732474O15 gene |
| BI144810 | CDNA sequence BC024137, mRNA (cDNA clone IMAGE: 5136153), partial cds |
| BM238035 | Transcribed sequences |
| BB035414 | transmembrane protein 2 |
| NM_020002 | REC8-like 1 (yeast) |
| AU022985 | Transcribed sequences |
| BG066901 | Transcribed sequences |
| BG086638 | H3128G05-5 NIA Mouse 15K cDNA Clone Set Mus musculus cDNA clone H3128G05 |
| | 5′, mRNA sequence. |
| M69069 | histocompatibility 2, K region |
| BM202761 | Transcribed sequence with moderate similarity to protein ref: NP_081764.1 |
| | (M. musculus) RIKEN cDNA 5730493B19 [Mus musculus] |
| AK005477 | dihydrolipoamide S-succinyltransferase (E2 component of 2-oxo-glutarate complex) |
| NM_008050 | synonyms: ELP, SF1, SF-1, Ad4BP, ELP-3, Ftzf1, Ftz-F1; This sequence comes |
| | from FIG. 5; fushi tarazu 1 factor homolog; steroidogenic factor 1; adrenal 4-binding |
| | protein; go_component: nucleus [goid 0005634] [evidence IDA] [pmid 12861022]; |
| | go_function: protein binding [goid 0005515] [evidence IPI] [pmid 12732652]; |
| | go_function: transcription factor activity [goid 0003700] [evidence IEA]; go_function: |
| | steroid hormone receptor activity [goid 0003707] [evidence IEA]; go_function: ligand- |
| | dependent nuclear receptor activity [goid 0004879] [evidence IEA]; go_function: |
| | receptor activity [goid 0004872] [evidence IEA]; go_function: DNA binding [goid |
| | 0003677] [evidence IDA] [pmid 12730328]; go_function: transcriptional activator |
| | activity [goid 0016563] [evidence IDA] [pmid 12730328]; go_process: regulation of |
| | transcription, DNA-dependent [goid 0006355] [evidence IDA] [pmid 12651892]; |
| | go_process: positive regulation of transcription from Pol II promoter [goid 0045944] |
| | [evidence IDA] [pmid 12732652]; go_process: transcription [goid 0006350] [evidence |
| BG076300 | H3158A12-3 NIA Mouse 15K cDNA Clone Set Mus musculus cDNA clone H3158A12 |
| | 3′, mRNA sequence. |
| NM_024182 | RIO kinase 3 (yeast) |
| U46151 | t-complex-associated testis expressed 2 |
| BB558081 | Transcribed sequences |
| BB363201 | RIKEN cDNA 4930418P06 gene |
| AJ437646 | defensin beta 11 |
| AW495116 | Transcribed sequence with weak similarity to protein pir: S23650 (H. sapiens) S23650 |
| | retrovirus-related hypothetical protein II - human retrotransposon LINE-1 |
| BB039251 | 13 days embryo male testis cDNA, RIKEN full-length enriched library, |
| | clone: 6030441H14 product: unknown EST, full insert sequence |
| AV260760 | AV260760 RIKEN full-length enriched, adult male testis (DH10B) Mus musculus |
| | cDNA clone 4930415C23 3′, mRNA sequence. |
| U83636 | nuclear antigen Sp100 |
| AK010834 | RIKEN cDNA 2410193C02 gene |
| BG070666 | Transcribed sequences |
| BB233495 | BB233495 RIKEN full-length enriched, 3 days neonate thymus Mus musculus cDNA |
| | clone A630043O08 3′, mRNA sequence. |
| BC016468 | elongation of very long chain fatty acids (FEN1/Elo2, SUR4/Elo3, yeast)-like 3 |
| BM933567 | Transcribed sequence with moderate similarity to protein sp: P00722 (E. coli) |
| | BGAL_ECOLI Beta-galactosidase |
| BB053811 | BB053811 RIKEN full-length enriched, 12 days embryo male wolffian duct Mus |
| | musculus cDNA clone 6720466E23 3′, mRNA sequence. |
| AK005115 | TRAF-binding protein |
| BB343867 | Transcribed sequences |
| BB554189 | arachidonate 12-lipoxygenase |
| BM933224 | Transcribed sequences |
| AK014942 | cation channel, sperm associated 3 |
| NM_016923 | lymphocyte antigen 96 |
| BB083532 | Adult male diencephalon cDNA, RIKEN full-length enriched library, clone: 9330183F02 |
| | product: hypothetical protein, full insert sequence |
| BB155332 | 16 days neonate thymus cDNA, RIKEN full-length enriched library, |
| | clone: A130024J23 product: unknown EST, full insert sequence |
| NM_026184 | RIKEN cDNA 1300013B24 gene |
| NM_007423 | albumin 1 |
| M29881 | histocompatibility 2, Q region locus 7 |
| AK018259 | Adult male medulla oblongata cDNA, RIKEN full-length enriched library, |
| | clone: 6330582A15 product: unclassifiable, full insert sequence |
| NM_010051 | dickkopf homolog 1 (Xenopus laevis) |
| AF462069 | N-acetylglutamate synthase |
| BB086994 | frizzled homolog 8 (Drosophila) |
| AF119253 | histocompatibility 2, class II antigen A, alpha |
| NM_021454 | CDC42 effector protein (Rho GTPase binding) 5 |
| AI643890 | RIKEN cDNA B130017I01 gene |
| BB830629 | Transcribed sequences |
| AI464581 | Transcribed sequences |
| NM_010432 | homeodomain interacting protein kinase 1 |
| BG071681 | RIKEN cDNA 9130023F12 gene |
| AV205672 | AV205672 RIKEN full-length enriched, adult male testis Mus musculus cDNA clone |
| | 1700081H04 3′, mRNA sequence. |
| BF136544 | fibrinogen-like protein 2 |
| BB543054 | 0 day neonate eyeball cDNA, RIKEN full-length enriched library, clone: E130202F10 |
| | product: hypothetical protein, full insert sequence |
| BB232349 | Transcribed sequences |
| AV264531 | AV264531 RIKEN full-length enriched, adult male testis (DH10B) Mus musculus |
| | cDNA clone 4930500F18 3′, mRNA sequence. |
| BF152877 | Transcribed sequences |
| BM117570 | Transcribed sequences |
| AV351492 | RIKEN cDNA 2610037M15 gene |
| BB216909 | BB216909 RIKEN full-length enriched, adult male aorta and vein Mus musculus |
| | cDNA clone A530039O16 3′, mRNA sequence. |
| BM213120 | Transcribed sequences |
| AV273072 | eyes absent 3 homolog (Drosophila) |
| AU067780 | RIKEN cDNA 5330421F07 gene |
| BG067463 | amyloid beta (A4) precursor protein-binding, family B, member 2 |
| AU021791 | Transcribed sequences |
| AF153199 | matrix metalloproteinase 19 |
| BB338428 | 0 day neonate eyeball cDNA, RIKEN full-length enriched library, clone: E130202F10 |
| | product: hypothetical protein, full insert sequence |
| BB326058 | cholinergic receptor, nicotinic, alpha polypeptide 5 |
| X00246 | histocompatibility 2, D region locus 1 |
| BB368667 | RIKEN cDNA C130044A18 gene |
| AK005688 | DnaJ (Hsp40) homolog, subfamily B, member 3 |
| J00406 | histocompatibility 2, K region |
| M83244 | histocompatibility 2, D region locus 1 |
| BB541505 | 0 day neonate eyeball cDNA, RIKEN full-length enriched library, clone: E130114J20 |
| | product: unknown EST, full insert sequence |

TABLE 3

| Genbank No. | Gene Title |
|---|---|
| AV328280 | solute carrier family 1 (glial high affinity glutamate |
| | transporter), member 2 |
| NM_013626 | peptidylglycine alpha-amidating monooxygenase |
| NM_007652 | CD59a antigen |
| BB251739 | BB251739 RIKEN full-length enriched, 7 days neonate |
| | cerebellum Mus musculus cDNA clone A730047M15 3′ |
| | similar to D29766 Rattus norvegicus mRNA for |
| | Crk-associated substrate, p130, mRNA sequence. |
| BB524597 | Kruppel-like factor 7 (ubiquitous) |
| AA408781 | DNA segment, Chr 5, Brigham & Women's Genetics 1524 |
| | expressed |
| BB392041 | BB392041 RIKEN ful-length enriched, 0 day neonate |
| | cerebellum Mus musculus cDNA clone C230077J16 3′, |
| | mRNA sequence. |
| W45978 | Clone IMAGE: 4206343, mRNA |

TABLE 4

| Genbank No. | Gene Title |
|---|---|
| BG065782 | cysteine-rich hydrophobic domain 1 |
| BB181247 | myelin basic protein |
| AV223474 | AV223474 RIKEN full-length enriched, 18 days pregnant, |
| | placenta and extra embryonic tissue Mus musculus cDNA |
| | clone 3830411J16 3′, mRNA sequence. |
| AK010621 | RIKEN cDNA 2410030J07 gene |
| AV361868 | AV361868 RIKEN full-length enriched, adult male corpus |
| | striatum Mus musculus cDNA clone 7630402M06 3′, |
| | mRNA sequence. |
| BB234940 | discoidin domain receptor family, member 1 |
| AV156640 | AV156640 Mus musculus head C57BL/6J 12-day embryo |
| | Mus musculus cDNA clone 3000003N22, mRNA sequence. |
| BC020004 | G protein-coupled receptor, family C, group 5, member B |
| AV344962 | AV344962 RIKEN full-length enriched, adult male |
| | olfactory bulb Mus musculus cDNA clone |
| | 6430548L14 3′, mRNA sequence. |
| BI328541 | kinesin family member 5B |

In the above genes, for example, kinesin family member 5B (GenBank No. BI328541), cysteine-rich hydrophobic domain 1 (GenBank No. BG065782), solute carrier family 1 (glial high affinity glutamate transporter), member 2 (GenBank No. AV328280) described in Tables 3 and 4 are the genes predicted to be associated with the abnormal behaviors and mental diseases.

The medicament screening can be performed by evaluating the increase or decrease of at least one gene listed in the above Tables 1 to 4 and predicted to be closely associated with nerve cell functions, the abnormal behaviors and the mental diseases by administering the medicament candidate compound in the screening method of the present invention.

### EXAMPLES

The present invention will be described in more detail below using Examples.

### Reference Example 1

DNA Fragment of Non-Cleavable proBDNF

A gene recombination experiment was performed for introducing mutations M (Met at position 125) and L (Leu at position 127) at two R (Lys at positions 125 and 127) sites present proximally to a cleavage site (downward arrow) of a prodomain of proBDNF based on SNP database in NCBI.

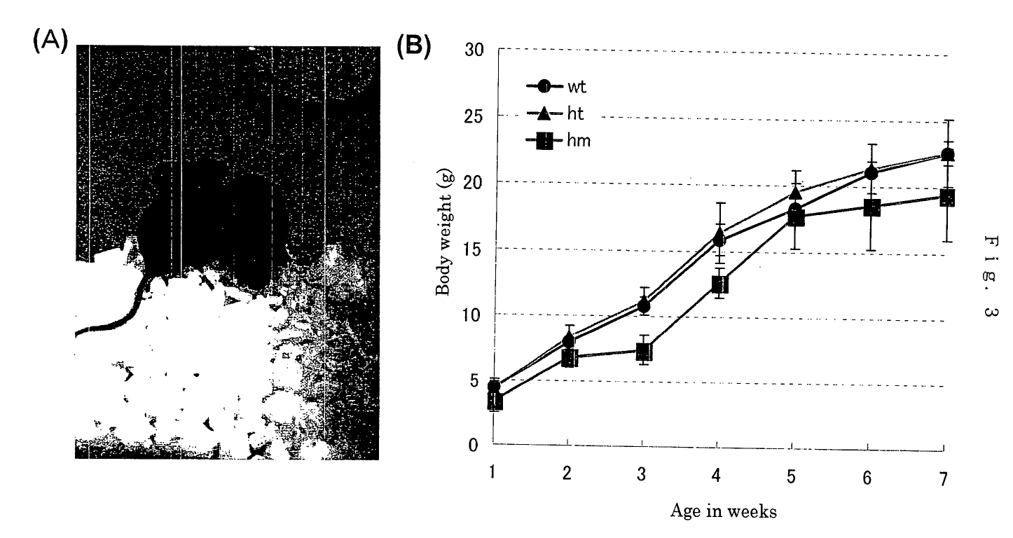

A DNA fragment corresponding to the amino acid sequence (SEQ ID NO:1) in FIG. 2 using DNA prepared from human blood was amplified by PCR, and cloned to TA vector (Invitrogen). In order to obtain the gene in which two R (Lys, capital letter, at positions 125 and 127) have been mutated to M and L, respectively, using primers, GCAAACATGTCCATGATGGTCCTGCGCCACTCTGACCC (primer 1, SEQ ID NO:4) and GGGTCAGAGTGGCGCAGGACCATCATGGACATGTTTGC (primer 2, SEQ ID NO:5) and Quick-change XL site-directed mutagenesis kit (Stratagene), an objective non-cleavable proBDNF (BDNF-ML) was obtained.

### Example 1

(Materials and Methods)

Construction of Knockin Vector and Production of Knockin Mice

In order to isolate DNA fragments of a long arm and a short arm in the knockin vector (FIG. 1), PCR was performed using a lambda DASHII (Stratagene, CA) genomic library of genomic cDNA prepared from ES cells D3 derived from 129/Sv mouse strain as a template.

The long arm (DNA data base No. AY057907: 49015-54460) was amplified by PCR using the primer 1 (GTCGACCTTCCACACTCCTACTAGG, SEQ ID NO:6) and the primer 2 (ATCGATCACTCTTCTCACCTGGTGG, SEQ ID NO:7), and cloned to the TA vector (Invitrogen).

The short arm (DNA data base No. AY057907: 49015-54460) was likewise cloned using the primer 3 (TTAATTAATGGATTTATGTTGTATAGATTATATTGAG, SEQ ID NO:8) and the primer 4 (GGCGCGCCCAGCGGCTTGGCAGCCGTCACGG, SEQ ID NO:9).

In order to add a myc tag sequence at C terminus of the DNA fragment of the non-cleavable proBDNF (Reference Example 1), PCR was performed using the primer 5 (ATCGATCACTCTTCTCACCTGGTGG, SEQ ID NO:10) and the primer 6 (GCGGCCGCCTATCTTCCCCTTTTAATGG, SEQ ID NO:11), and the resulting fragment was cloned to the TA vector (Invitrogen) (mutant BDNF in FIG. 1).

A knockin vector (FIG. 1) for producing the mouse that expresses a precursor form of BDNF (proBDNF) was made by introducing a SalI-ClaI fragment of the long arm (SEQ ID NO:10: SalI and ClaI sites had been added in the primers 1 and 2, respectively), a PacI-AscI fragment of the short arm (SEQ ID NO:11: PacI and AscI sites had been added in the primers 3 and 4, respectively), and a ClaI-NotI fragment encoding the non-cleavable proBDNF into a targeting vector pMulti-ND 1.0 (Inoue et al., Nature, vol. 434, 234-237, 2005).

The knockin mice were produced in accordance with the method previously described (Inoue et al., Nature, vol. 434, 234-237, 2005). That is, using the constructed vector to establish ES cells having the mutation in genomic BDNF, the ES cells were used to produce the mice containing the mutant gene in homozygote and heterozygote. That is, the mutant gene was introduced into the ES cells (ES cell line D3 derived from 129/Sv strain) by electroporation, and the ES cells having the mutant gene were established by negative selection with neomycin resistant gene (FIG. 1, neo^{r}) in the presence of 100 μg of neomycin and positive selection with diphtheria toxin (FIG. 1, DT). Blastocysts were collected from C57BL/6 mice, and the ES cells in which the mutant gene had been introduced were injected into the blastocysts by microinjection. The blastocysts in which the ES cells had been injected were transplanted in uterus of a pseudopregnant mouse (ICR strain) obtained by crossing with a male mouse that had undergone vasoligature. Heterozygous mice were obtained by delivery of the pseudopregnant mouse. Cre-lox recombinase was added as a lox sequence to both ends of a neo gene in the targeting vector pMulti-ND 1.0 to remove the neo gene. The neo gene was removed by crossing the heterozygous mouse with B6.Cg-Tg(CAG-cre)CZ-MO2Osb, a mouse expressing Cre-lox recombinase to produce a new heterozygous mouse, which was the first generation. These heterozygous mice were crossed one another to produce homozygous mice which were the second generation. The genotypes of the mice were identified by PCR and Southern blotting. Fertilized eggs of the resulting knockin mice were deposited as “Accession number FERM P-20742” in International Patent Organism Depositary, National Institute of Advanced Industrial Science and Technology as of Dec. 21, 2005.

(Analysis of Produced Knockin Mice)

Genotype of Resulting Knockin Mice

The genotypes of progenies derived from heterozygote crossing are shown in Table 5.

TABLE 5

| | | Genotype | | | |
|---|---|---|---|---|---|
| Genotype | Age | +/+ | +/− | −/− | Total |
| Mutant BDNF | neonate | 4(20) | 6(55) | 5(25) | 20 |

The number of each genotype (+/+: wild type homozygote; +/−: heterozygote; −/−: knockin homozygote) of neonatal mice is shown.
The number in a parenthesis shows a frequency (%) of each genotype.

In knockin homozygous mice at age of 2 to 3 weeks, the following phenotypes which were not observed in the wild type homozygotes were observed. They are (1) rolling when started to walk, (2) staggering gait, (3) thrashing with lying upside down and (4) lowering of a lower body in an attitude. The heterozygotes tended to exhibit the intermediate phenotypes between the knockin homozygotes and the wild type homozygotes.

The knockin homozygous mice frequently tumbled with waddling on the 12th day after the birth, and on the 18th day, their quantity of motion was increased and they repeated the behavior of rolling and tumbling. On the 21st day, eyelids were opened and they ate foods by themselves. However, in the knockin homozygous mice, the quantity of motion was abnormally high, they were restless, and running and tumbling were repeated.

Such phenotypes in the knockin homozygous mice suggests abnormality of cerebellum, vestibule or inner ear, reduction of muscular force, temper tantrum, motor function disorder, learning disability (LD), ADHD (attention deficit/hyperactivity disorder), high-functioning autism and the like.

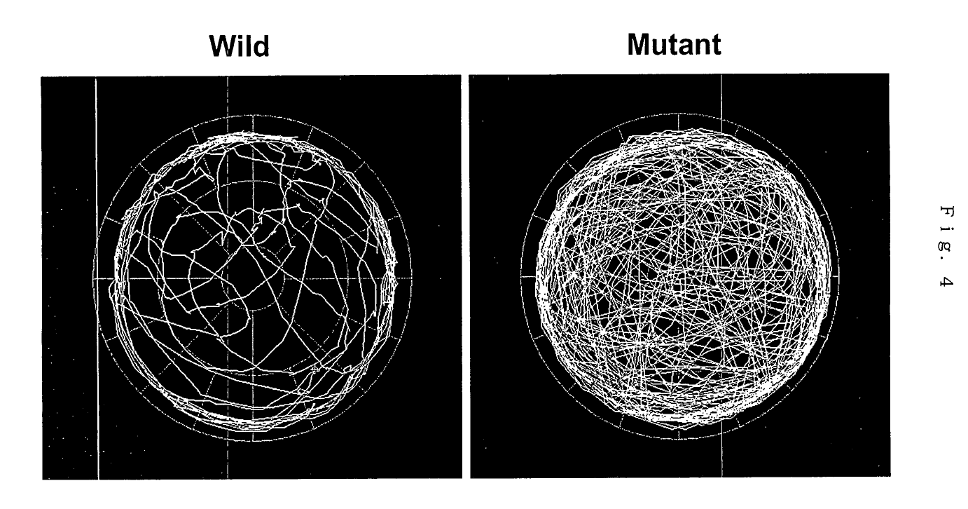

Although the knockin homozygous mice have such abnormalities, they are not lethal and grow with weight gain (FIG. 3), suggesting that the mental diseases in these mice are caused by functional modulation and structural abnormality in the nervous system.

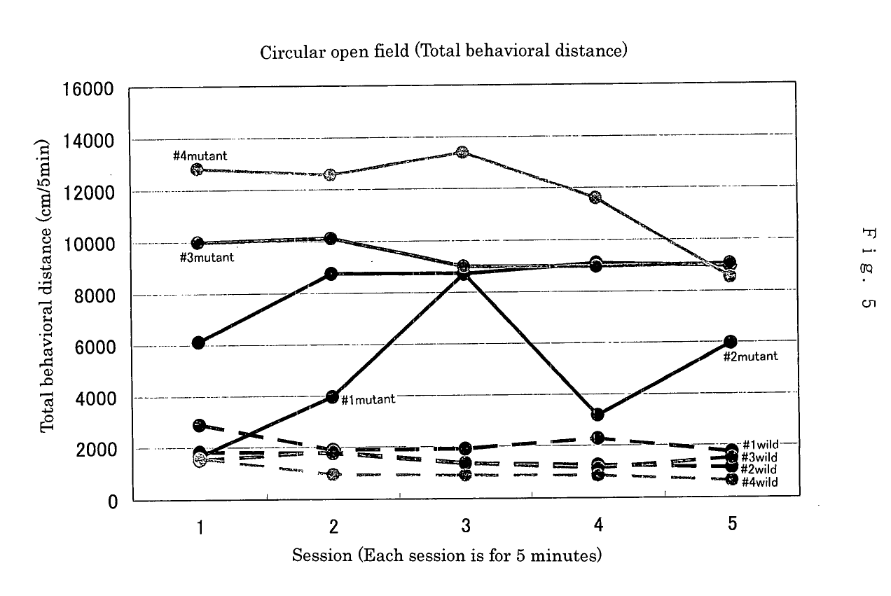

The abnormal behavior in the mutant BDNF gene-knockin mouse was quantitatively analyzed. An emotional change of the mouse is reflected to the quantity of activity and a behavioral pattern of the mouse when the mouse is placed in a novel environment. The mouse was placed in the novel environment which was an inexperienced circular field (a diameter of 50 cm and a wall height of 40 cm in the field) for 25 to 30 minutes, and the quantity of activity in the meantime was compared between the wild type homozygous mice (wild) and the knockin homozygous mice (mutants). The mouse was placed 30 minutes before the experiment in a room in which the circular field had been disposed, and placed calmly in the circular field using a paper box. The activity of the mouse in the circular field was measured by recording using DV-Track video tracking system supplied from Muromachi Kikai. FIG. 4 shows a representative trace of the behavior of the mouse placed in the open field for 5 minutes, and the mutant mouse shows the remarkable quantity of activity compared with the wild type mouse.

The mouse placed in the novel environment is adapted to the environment with time by searching behavior. In FIG. 5, the quantity of activity every 5 minutes for 25 minutes after being placed in the open field was plotted for the wild type mouse and the mutant mouse. The wild type mouse was adapted to the circular field and gradually shortened its behavioral distance, but in the mutant mouse, such an adaptability was not observed and its activity was continued. Therefore, the mutant BDNF gene-knockin mouse is hardly adapted remarkably to the novel environment.

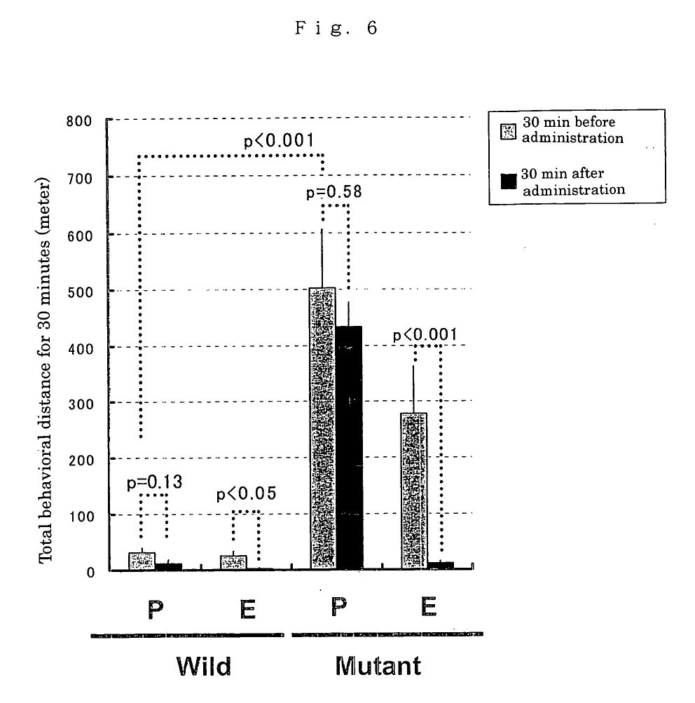

However, it was found that such a disorder was improved by administration of an anxiolytic drug, etizolam (FIG. 6). Etizolam is a short term action type GABA, a neurotransmission accelerator (reference: Elder C. A. et al.: Drug Metab. Dispos., vol. 23, 776, 1995). In the experiment, Depas tablets (Mitsubishi Pharma Corporation) containing etizolam were dissolved in PBS, and its supernatant was used as an etizolam solution. A protocol was comprising (I) measuring the total quantity of activity in the mouse for 30 minutes after being placed in the open field, (II) intraperitoneally administering PBS or the etizolam solution (0.5 mg/kg) and (III) returning the mouse in the open field after 5 minutes and measuring the quantity of activity for 30 minutes in the mouse. The mice used were the wild type mice (wild) and the mutant mice (mutant) of littermates aged 4 to 5 weeks, and 4 to 5 mice were used per group. The experimental results in FIG. 6 are summarized. (I) The total behavioral distance for 30 minutes in the mutant mouse is 7 to 10 times longer than that in the wild type mouse (p<0.001). (II) Such an abnormal behavior in the mutant mouse is remarkably inhibited by the administration of the anxiolytic drug, etizolam (E in mutant group, p<0.001). However, PBS (P) which is a solvent of the etizolam solution is not effective (p=0.58). (III) Etizolam is certainly effective for the wild type mice (p<0.05), but PBS (P) is not effective (p=0.13). These results suggest that causes of the abnormal behavior in the mutant BDNF gene-knockin mice include mental anxiety. Therefore, the non-human knockin animal of the present invention is useful as a model animal for the diseases involving the mental anxiety, and can be suitably used for screening such diseases.

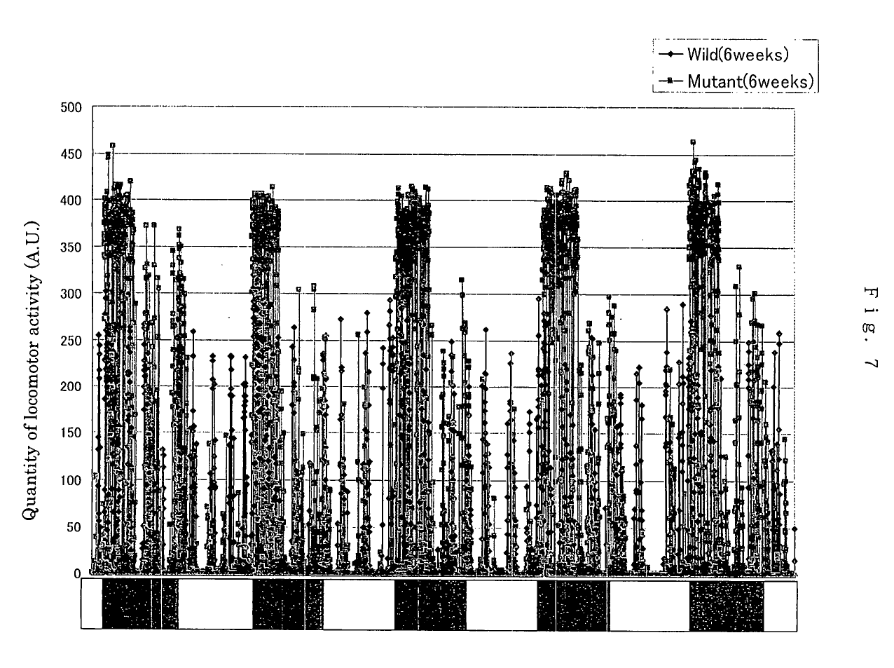

The abnormal behavior in the mutant BDNF gene-knockin mice was remarkable not only in the novel environment such as a circular field but also in a home cage. A locomotor activity measurement apparatus (Muromachi Kikai, Super-Mex) practically applying infrared ray was disposed above a breeding cage, and the quantity of locomotor activity in the home cage was measured for about 5 days (FIG. 7). The quantity of activity was represented by an arbitrary unit (A.U.). In FIG. 7, it was found that (i) the quantity of activity was higher in the mutant mice than in the wild type mice, (ii) the quantity of activity (A.U.) in the total measurement period was 3 times higher in the mutant mice than in the wild type mice, and (iii) the quantity of activity in the dark phase was 4 times or more higher in the mutant mice than in the wild type mice. From the above results, it is evident that the knockin homozygous mice have a neurologic symptom that they are restless in the usual breeding environment as well as in the novel environment.

The possibility is thought that tumbling and rolling frequently observed in a juvenile phase is caused by structural abnormality in cerebellum. Cerebellum morphology was compared by giving cresyl violet staining to mid sagital section of cerebellum for the knockin homozygous mice, heterozygous mice and the wild type mice aged 3 weeks (FIG. 8). As a result, in the mutant BDNF gene-knockin homologous mouse, the groove (white arrow) between VIb lobe and VII lobe and the groove (black arrow) of IX lobe disappeared. That is, it is suggested that the abnormal behavior in the mice is associated with the change in the brain structure.

Subsequently, in order to understand molecular basis concerning the abnormality in such mice, the expression of brain genes were analyzed in the mutant BDNF gene-knockin mice using DNA array (http://www.affymetrix.com). The whole brain was removed from the wild type mouse and the mutant homozygous mouse aged 5 weeks, and RNA was extracted therefrom. A one color method using GeneChip array (Affimetrix) was employed for the array analysis. As a result, genes whose expression amount had been increased to twice or more or decreased to 0.5 times or less were found in the mutant homozygous mouse compared with the wild type mouse as shown in Tables 1 to 4. The results of the array analysis suggest the presence of the molecular mechanism capable of explaining the neurological diseases in the mutant BDNF gene-knockin mice, and seems to be database helpful for study on etiology of the neurological diseases, study on the treatment, drug discovery and medicament screening elucidated from this mouse.

The knockin mouse of the present invention can be identified by genotyping in the following scheme.

Genomic DNA is purified by cutting 0.5 cm of a tail of a neonatal mouse and using DNeasy Tissue Kit (Qiagen). The purified DNA is dissolved in 200 μL of sterilized water. The knockin mouse is identified by PCR using synthetic primers having the following sequence.

Primer Sequences

| | |
|---|---|
| Primer #1: ATGACCATCCTTTTCCTTAC | (SEQ ID NO:12) |
| Primer #2: CTAATACTGTCACACACGC | (SEQ ID NO:13) |

PCR

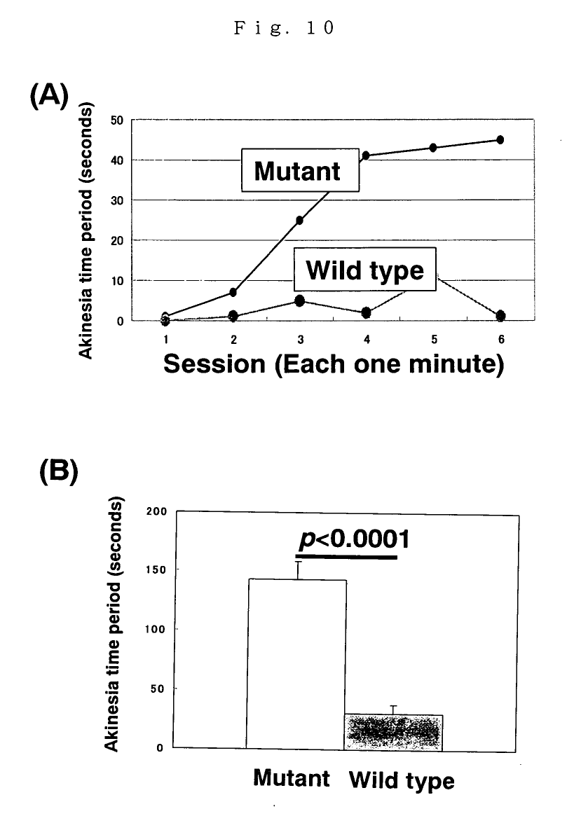

Using 20 μL of a reaction solution containing 0.5 μL pf purified DNA, 1 μL of Ex Taq (Takara), 2 μL of 10× buffer specific for Ex Taq (Takara), 1.6 (L of dNTPs mixture, 0.1 μL of 100 μM primer #1 solution and 0.1 μL of 100 μM primer #2 solution, PCR of reacting at 94° C. for one minute is performed, then a PCR cycle of reacting at 94° C. for 30 seconds, 60° C. for 30 seconds and 72° C. for 30 seconds is repeated 30 times, and finally the reaction at 72° C. for 2 minutes is performed. This PCR amplifies 435 bp of DNA derived from a knockin sequence and 440 bp of DNA derived from the mouse simultaneously. However, the DNA derived from the mouse is cleaved into 283 bp and 157 bp by a restriction enzyme, SmaI. This SmaI reaction is determined as in FIG. 9 by leaving stand 10 μL of a reaction solution containing 5 μL of the above PCR reaction solution, 1 μL of T buffer (Takara), 1 μL of 0.1% BSA and 1 μL of SmaI (Takara) at 37° C. for 2 hours and performing electrophoresis on 2% agarose gel.

Depressive Behavior in Mutant BDNF-Knockin Mice

The mental abnormality in the mutant BDNF-knockin mice was also remarkable in a tail suspension test. When the mouse is suspended by holding its tail, the mouse typically struggles to escape. However, the mouse gradually recognizes that it is not possible to escape, and exhibits the akinesia. The state in which this akinesia occupies the majority is defined as the depressive state, and the course up to this state is measured. The locomotor activity measurement apparatus (Muromachi Kikai, Super-Mex) practically applying the infrared ray was disposed in front of a tail suspension apparatus (Muromachi Kikai), and this course was measured as in FIG. 10. The experiment was performed in accordance with the reference (What's wrong with my mouse?, written by Jacqueline N. Crawley).

FIG. 10A shows representative transitions of an akinesia time period in the mutant mouse and the control mouse. Immediately after the mouse was suspended by the tail suspension apparatus, the measurement was started, and the akinesia time period was measured for 6 minutes. A vertical axis represents the akinesia time period in each session.

FIG. 10B shows the results obtained by measuring the akinesia time period for 6 minutes using 8 respective mice and statistically analyzing them. It was found that the akinesia time period in the mutant mice was significantly different from that in the wild type mice (t-test, p<0.0001), and that the akinesia time period in the mutant mice was about 5 times longer than that in the wild type mice. From the above results, it was found that the mutant BDNF knockin mice had the mental abnormality with remarkable depression.

1. A non-human knockin animal having a mutation in proBDNF gene wherein conversion from prOBDNF to BDNF has been inhibited by the mutation.

2. The non-human knockin animal according to claim 1 wherein said proBDNF gene having the mutation is any of the following (a) to (c):
(a) DNA comprising a base sequence represented by SEQ ID NO:3 or a complementary chain thereto;
(b) DNA which hybridizes with the DNA comprising the base sequence represented by SEQ ID NO:3 under a stringent condition and encodes proBDNF in which the conversion to BDNF has been inhibited; and
(c) DNA encoding an amino acid sequence having one or more amino acid substitutions, additions, deletions or insertions in an amino acid sequence represented by SEQ ID NO:2 and in which the conversion from proBDNF to BDNF has been inhibited.

3. The non-human knockin animal according to claim 1 wherein said animal is a mouse.

4. The non-human knockin animal according to claim 1 wherein a position 125 and/or 127 in the proBDNF gene has been mutated.

5. The non-human knockin animal according to claim 4 wherein the mutant proBDNF has mutations of R125M and R127L.

6. A vector for making a non-human knockin animal having a mutant proBDNF gene in which conversion from proBDNF to BDNF has been inhibited.

7. The vector according to claim 6 wherein the mutant proBDNF gene has been inserted in a position sandwiched with a long arm and a short arm.

8. A method for screening medicaments for at least one disease selected from abnormal behaviors and mental diseases, characterized in that a medicament candidate substance is administered to the non-human knockin animal according to any of claims 1 to 5.

9. The method according to claim 8 wherein the disease is at least one selected from the group consisting of diseases based on abnormality of cerebellum, vestibule or inner ear, diseases based on reduction of muscular force, temper tantrum, learning disability, ADHD (attention deficit/hyperactivity disorder), high-functioning autism, obsessive-compulsive disorder, schizophrenia, pervasive developmental disease, anorexia, dementia, sleep disturbance, panic disorder, neurosis, manic psychosis, depressive psychosis, bipolar disorder, psychosomatic disorder, anxiety disorders and intension.

10. The method for screening according to claim 8, characterized in that an efficacy of a medicament candidate compound is determined using it as an indicator whether an expressed amount of at least one gene which increases or decreases 2 times or more relative to that in a wild type animal with mutation of proBDNF gene comes close to an expressed amount of the wild type or not by administering the medicament candidate compound.


SEQUENCE LISTING (13 sequences)

SEQ ID NO: 1 - protein, 247 aa, Human
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 2 - protein, 247 aa, Artificial sequence
  Non-cleavable human proBDNF mutant
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMMVLRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 3 - DNA, 744 bp, Artificial sequence
  Non-cleavable human proBDNF mutant
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccataaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgatggtcctgcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 4 - DNA, 38 bp, Artificial sequence
  Primer for producing non-cleavable human proBDNF
gcaaacatgtccatgatggtcctgcgccactctgaccc

SEQ ID NO: 5 - DNA, 38 bp, Artificial sequence
  Primer for producing non-cleavable human proBDNF
gggtcagagtggcgcaggaccatcatggacatgtttgc

SEQ ID NO: 6 - DNA, 25 bp, Artificial sequence
  Primer for preparing long arm
gtcgaccttccacactcctactagg

SEQ ID NO: 7 - DNA, 25 bp, Artificial sequence
  Primer for preparing long arm
atcgatcactcttctcacctggtgg

SEQ ID NO: 8 - DNA, 37 bp, Artificial sequence
  Primer for preparing short arm
ttaattaatggatttatgttgtatagattatattgag

SEQ ID NO: 9 - DNA, 31 bp, Artificial sequence
  Primer for preparing short arm
ggcgcgcccagcggcttggcagccgtcacgg

SEQ ID NO: 10 - DNA, 25 bp, Artificial sequence
  Primer for introducing myc tag to C-terminal of non-cleavable human proBDNF
atcgatcactcttctcacctggtgg

SEQ ID NO: 11 - DNA, 28 bp, Artificial sequence
  Primer for introducing myc tag to C-terminal of non-cleavable human proBDNF
gcggccgcctatcttccccttttaatgg

SEQ ID NO: 12 - DNA, 20 bp, Artificial sequence
  Primer for genotyping of knock-in mouse
atgaccatccttttccttac

SEQ ID NO: 13 - DNA, 19 bp, Artificial sequence
  Primer for genotyping of knock-in mouse
ctaatactgtcacacacgc